(19) United States
(12) Patent Application Publication
(10) Pub. No.: US 2010/0048890 A1
(43) Pub. Date: Feb. 25, 2010

(54) CONJUGATED SMALL MOLECULES

(76) Inventors: Robert M. Grotzfeld, Carlsbad, CA (US); Zdravko V. Milanov, San Diego, CA (US); Hitesh K. Patel, Encinitas, CA (US); Andiliy G. Lai, San Diego, CA (US); Shamal A. Mehta, San Diego, CA (US); David J. Lockhart, Del Mar, CA (US)

Correspondence Address: JONES DAY, 222 EAST 41ST ST, NEW YORK, NY, 10017, US

(21) Appl. No.: 12/495,526

(22) Filed: Jun. 30, 2009

Related U.S. Application Data
(63) Continuation of application No. 11/030,638, filed on Jan. 6, 2005, now Pat. No. 7,355,104.
(60) Provisional application No. 60/535,173, filed on Jan. 7, 2004.
(60) Provisional application No. 60/557,941, filed on Mar. 30, 2004.

(51) Int. Cl. C07D 498/22; C07D 473/16; C07D 471/04

(52) U.S. Cl. 540/545; 544/277; 544/279

(57) ABSTRACT

Provided herein are linker compounds and conjugates that include the linker compounds. In one embodiment, the linker compounds comprise 2 or 3 residues of 6-aminohexanoic acid and optionally 7-10 residues of polyethyleneglycol (PEG). The linker compounds are useful in forming conjugates with one or more components useful in biopharmaceutical or bioanalytical applications. In particular, the biopharmaceutically useful compounds are kinase inhibitors. The conjugates described herein have utility in a variety of diagnostic, separation, and therapeutic applications.

### CROSS REFERENCE TO RELATED APPLICATIONS

This application a continuation of U.S. application Ser. No. 11/031,638, filed Jan. 7, 2005, which claims the benefit of Provisional Application U.S. Ser. No. 60/535,173, filed Jan. 7, 2004 and Provisional Application U.S. Ser. No. 60/557,941, filed Mar. 30, 2004. The entire contents of each of which are incorporated by reference in their entirety for all purposes.

### BACKGROUND OF THE INVENTION

Bioanalytical or biopharmaceutical applications often require that compounds and biological molecules be coupled to other compounds or molecules to form a conjugate. For example, “immunoconjugate” generally refers to a conjugate composed of an antibody or antibody fragment and some other molecule such as a label compound (e.g., a fluorophore), a binding ligand (e.g., a biotin derivative), or a therapeutic agent (e.g., a therapeutic protein or toxin). These particular conjugates are useful in reporting the presence of the antibody, binding or capturing the antibody, and targeting the delivery of a therapeutic agent to a specific site, respectively.

Typically, conjugates are prepared by covalently coupling one of the conjugate components to the other. For example, the immunoconjugate referenced above may be prepared by coupling a label compound, a binding ligand, or a therapeutic agent to an antibody or antibody fragment. Often the coupling involves the use of a linker compound or molecule which serves to join the conjugate components. Typically, the linker is selected to provide a stable coupling between the two components, and to control the length and/or the geometry over which he interaction can occur.

For example, biotin conjugates are widely used in biological sciences. Biotin is a naturally occurring vitamin which has an extremely high binding affinity (K_{d}≈10⁻¹⁵ M⁻¹) for avidin and streptavidin. Because of the affinity of biotin for avidin, biotin-containing conjugates have been widely used in bioanalytical procedures including immunoassays, affinity chromatography, immunocytochemistry, and nucleic acid hybridization (Wilchek and Bayer, Meth. Enzymol. 184:5, 1990). Bioanalytical assays often take advantage of the high binding affinity of biotin for avidin through the covalent coupling of biotin to one of the assay components. Biotin may be covalently coupled to many different types of molecules, including proteins, such as antibodies, antibody fragments, enzymes and hormones; nucleic acids such as oligonucleotides and a nucleic acid probes; and smaller molecules such as drugs or other similar compounds. Moreover, in some applications biotin may be coupled to a solid phase or support.

The covalent coupling of biotin to another molecule involves bond formation through chemical reaction between suitable chemical functional groups and a reactive biotin derivative. Reactive biotin derivatives for conjugation can be prepared from biotin, and are most commonly carboxylic acid derivatives, amines, or hydrazide derivatives. Common reactive biotin derivatives include reactive biotin esters such as an N-hydroxysuccinimide (NHS) ester, and biotin hydrazide. Alternatively, reactive biotin derivatives can be obtained from commercial sources including Sigma (St. Louis, Mo.), Pierce (Rockford, Ill.), Molecular Biosciences (Boulder, Colo.), and Molecular Probes (Eugene, Oreg.). Methods of conjugating biotin derivatives to proteins have been described in numerous publications (Harlow and Lane, Antibodies: A Laboratory Manual, NY: Cold Spring Harbor Laboratory, 1988, pp. 340-341, and Rose et al., Bioconjug. Chem. 2 154, 1991).

In addition to biotin, other compounds are commonly coupled to biological molecules for use in bioanalytical procedures. Typically, these compounds are useful in labeling the biological molecule for detection purposes. Common labeling compounds include fluorescent dyes, as well as ligands for binding to their respective binding partners. Examples of common fluorescent dyes used for this purpose include fluorescein and rhodamine, and examples of ligands for binding to their binding partners include drug compounds such as digoxigenin and β-lactam antibiotics. Numerous other compounds suitable for use as labels in specific binding techniques have also been described in the literature. Like biotin, these compounds are generally derivatized to contain Functional groups that react readily with the biological molecule. For example, fluorescein isothiocyanate is a reactive fluorescein derivative which may readily be conjugated to proteins through their sulfhydryl groups. Furthermore, the attachment of a tether containing thiol or polyhistidine functionalities allows a molecule of interest to be bound to a solid surface, such as, for example, gold or nickel surfaces.

Effective conjugation of a compound, such as biotin or a fluorescent dye, to a biological molecule generally requires that the resulting labeled conjugate retain the bioactivity of the biological molecule. A conjugate may have only limited utility if, upon coupling, the functional activity of the biological molecule is diminished or lost. For example, for an antibody conjugate, retention of antigen binding activity (immunoreactivity) is of foremost importance. Because some antibodies lose immunoreactivity upon labeling of their free amino groups, presumably due to the presence of these groups in the antigen binding site of the antibody, the site or sites at which a label is attached to a biological molecule is of considerable importance. Similarly, some enzymes contain free amino groups in their active sites which, upon their use as a labeling site, may result in a loss of enzymatic activity. Many enzymes also contain sulfhydryl groups in their active sites and are inactivated by labeling with sulfhydryl-reactive compounds such as fluorescein isothiocyanate.

In addition to retaining bioactivity, the stability of the conjugate with respect to linkage of the compound to the biological molecule is also important. For example, loss of a label from a conjugate typically results in the loss of ability to follow the conjugate in a bioanalytical procedure. In an attempt to provide stable linkages, conjugates are often coupled through amide and hydrazone bonds. Amide linkages are formed by reaction between an amino group and a carboxylic acid group, and hydrazone linkages result from reaction of a carbonyl group (such as an aldehyde group) and a hydrazine group. The relatively high stability of these linkages at neutral pH has led to their wide use in conjugation techniques. However, these linkages are not flexible enough to allow control over the distance between the components and to control the hydrophobicity and hydrophilicity of the conjugates. In addition to amide linkages, other functional groups may be employed to couple the molecule of interest and the linkers. For example, alcohols and phenols can be coupled via ether or urethane groups, amines can be alkylated or converted to ureas, aryl halides can be linked by various carbon-carbon coupling methods, e.g. Heck or Stille coupling.

Accordingly, there is a need in the art for improved linkages for conjugating a biological molecule with, for example, a label compound, a binding ligand or agent, or a therapeutic agent. Such linkages preferably have enhanced stability and control the length between the biological molecules.

### SUMMARY OF THE INVENTION

The present invention provides linker compounds and stably-linked conjugates. The stably-linked conjugates of the invention find use in the immunodiagnostic field, in separation techniques, in drug discovery, assay development, screening, and as therapeutics.

In one aspect are compounds comprising the structure of FORMULA A1:

A-(X₁—(CR₁R₂)ₚ—C(═X₂))ₙ—X₃-T₁-(CR₃R₄X₄CR₅R₆)ₘ-T₂-L-B (FORMULA Al)

wherein A and B are independently selected functional groups capable of forming a covalent linkage or a component useful in biopharmaceutical or bioanalytical applications;
X₁, X₂, X₃, and X₄ are independently selected from the group consisting of O, S, and NH;
   R₁, R₂, R₃, R₄, R₅, R₆, R₇, and R₈, are independently selected from the group consisting of hydrogen, halogen, or lower alkyl;
T₁ and T₂ are independently selected from the group consisting of (CH₂)ᵣ, a bond or triazole;
L is a bond or a moiety comprising a —NH— group;
p is 1, 2, 4, 4, 5, or 6;
n is 0, 1, 2, 3, 4, 5, or 6;
r is 1, 2, 3, 4, 5, or 6; and
m is equal to or greater than 3.

In a further embodiment of compounds having the structure of FORMULA A1, A and B are independently selected from the group consisting of a carboxylic acid group, an amine group, an aminoxy group, a hydrazide group, a semicarbazide group, a hydroxyl group, a thiol group, an isocyanate group, a thioisocyanate group, a maleimide group, a halide, azide, a boronic acid derivative and a carboxylic acid derivative.

In a further or additional embodiment of compounds having the structure of FORMULA A1 or any of the embodiments of compounds having the structure of FORMULA A1, A is biotin, a biotinyl group or a derivative of a biotinyl group.

In a further or additional embodiment of compounds having the structure of FORMULA A1 or any of the embodiments of compounds having the structure of FORMULA A1, X₄ is O. In a further or additional embodiment of compounds having the structure of FORMULA A1 or any of the embodiments of compounds having the structure of FORMULA A1, X₂ is O.

In a further or additional embodiment of compounds having the structure of FORMULA A1 or any of the embodiments of compounds having the structure of FORMULA A1, R₁, R₂, R₃, R₄, R₅, and R₆ are hydrogen. In a further or additional embodiment of compounds having the structure of FORMULA A1 or any of the embodiments of compounds having the structure of FORMULA A1, p is 5; n is 1, 2, or 3; and m is 6, 7, 8, 9, or 10.

In a further or additional embodiment of compounds having the structure of FORMULA A1 or any of the embodiments of compounds having the structure of FORMULA A1, T₂ is CH₂. In an alternative embodiment of compounds having the structure of FORMULA A1 or any of the embodiments of compounds having the structure of FORMULA A1, T₁ is a triazole.

In a further or additional embodiment of compounds having the structure of FORMULA A1 or any of the embodiments of compounds having the structure of FORMULA A1, L is a direct bond. In an alternative embodiment of compounds having the structure of FORMULA A1 or any of the embodiments of compounds having the structure of FORMULA A1, L is a moiety comprising an —NH— group. In a further embodiment of compounds having the structure of FORMULA A1 or any of the embodiments of compounds having the structure of FORMULA A1, L is a moiety comprising an —NH—C(O)— group. In a further embodiment of compounds having the structure of FORMULA A1 or any of the embodiments of compounds having the structure of FORMULA A1, L is selected from the group consisting of —NH—, —C(O)NH—, —NHC(O)NH—, and —C(O)CH₂CH₂C(O)NH—.

In a further or additional embodiment of compounds having the structure of FORMULA A1 or any of the embodiments of compounds having the structure of FORMULA A1, A is a component useful in biopharmaceutical or bioanalytical applications. In a further or additional embodiment of compounds having the structure of FORMULA A1 or any of the embodiments of compounds having the structure of FORMULA A1, B is a component useful in biopharmaceutical or bioanalytical applications. In a further or additional embodiment of compounds having the structure of FORMULA A1 or any of the embodiments of compounds having the structure of FORMULA A1, at least one of A or B is a binding agent, a label compound, or a therapeutic agent. In a further or additional embodiment of compounds having the structure of FORMULA A1 or any of the embodiments of compounds having the structure of FORMULA A1, A or B is a binding agent selected from the group consisting of biotin, antigen, antibody, riboflavin, cytostatin, and val-phosphate. In a further or additional embodiment of compounds having the structure of FORMULA A1 or any of the embodiments of compounds having the structure of FORMULA A1, A or B is a therapeutic agent selected from the group consisting of spiramycin, ipriflavone, mesalazine, and crotamiton. In a further or additional embodiment of compounds having the structure of FORMULA A1 or any of the embodiments of compounds having the structure of FORMULA A1, A or B is a label compound selected from the group consisting of a fluorescent label, an enzyme, an enzyme substrate, and a radioactive label. In a further or additional embodiment of compounds having the structure of FORMULA A1 or any of the embodiments of compounds having the structure of FORMULA A1, A or B is a fluorescent label selected from fluorescein, rhodamine, FITC (fluorescein isothiocyanate), HEX (4,5,2′,4′,5′,7′-hexachloro-6-carboxyfluorescein), 5-IAF, TAMRA (6-carboxytetramethylrhodamine), TET (4,7,2′,7′-tetrachloro-6-carboxyfluorescein), XRITC (rhodamine-X-isothiocyanate), texas red, CY2, CY3 and CY5. In a further or additional embodiment of compounds having the structure of FORMULA A1 or any of the embodiments of compounds having the structure of FORMULA A1, one of A or B is an enzyme selected from alkaline phosphatase, horseradish peroxidase, β-galactosidase and luciferase. In a further or additional embodiment of compounds having the structure of FORMULA A1 or any of the embodiments of compounds having the structure of FORMULA A1, at least one of A or B is a kinase inhibitor. In a further or additional embodiment of compounds having the structure of FORMULA A1 or any of the embodiments of compounds having the structure of FORMULA A1, at least one of A or B is a binding agent or a label compound and the other of A or B is a therapeutic agent. In a further or additional embodiment of compounds having the structure of FORMULA A1 or any of the embodiments of compounds having the structure of FORMULA A1, A or B is a therapeutic agent that is a kinase inhibitor. In a further or additional embodiment of compounds having the structure of FORMULA A1 or any of the embodiments of compounds having the structure of FORMULA A1, A or B is a kinase inhibitor selected from the group consisting of:

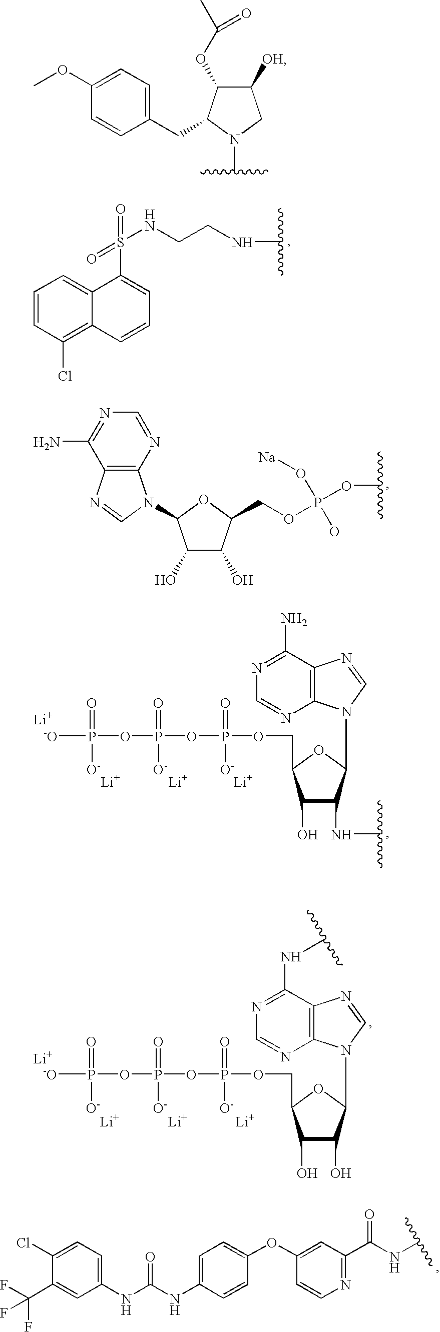

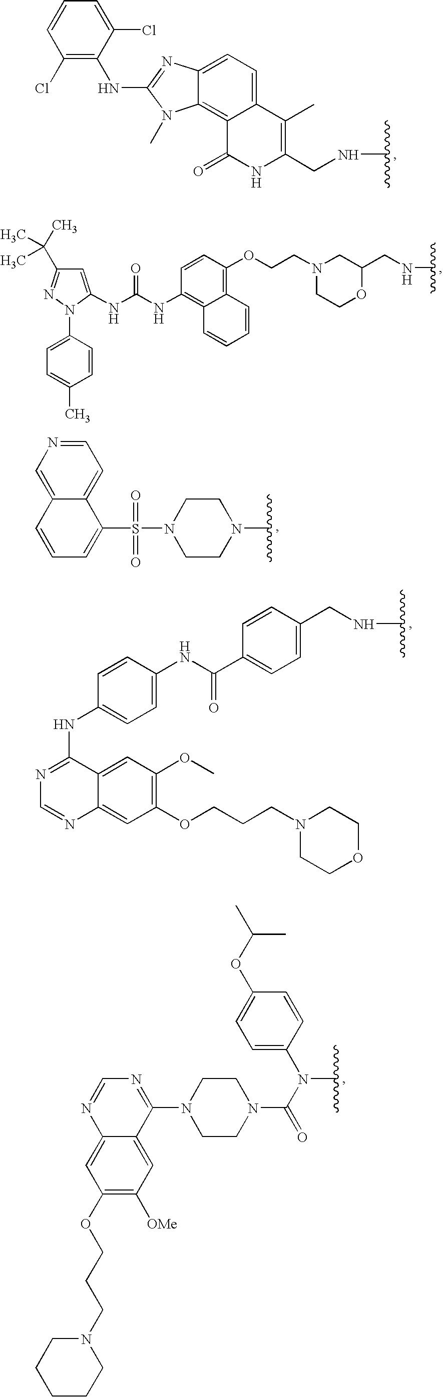

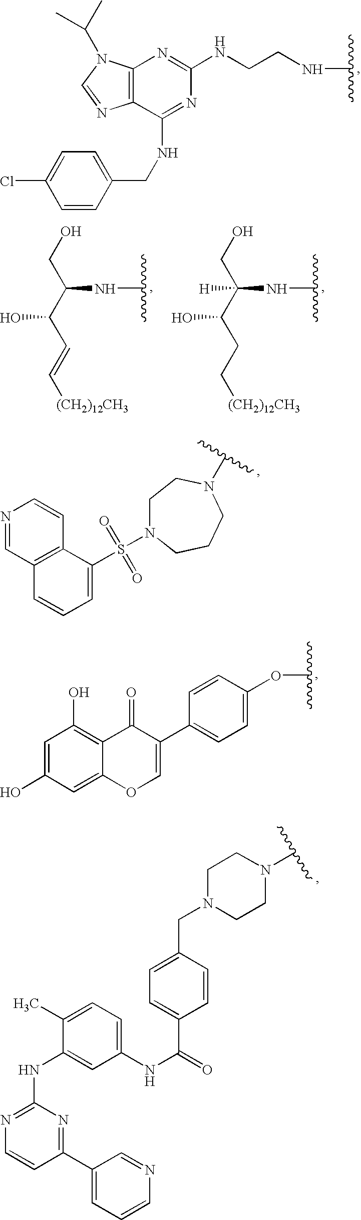

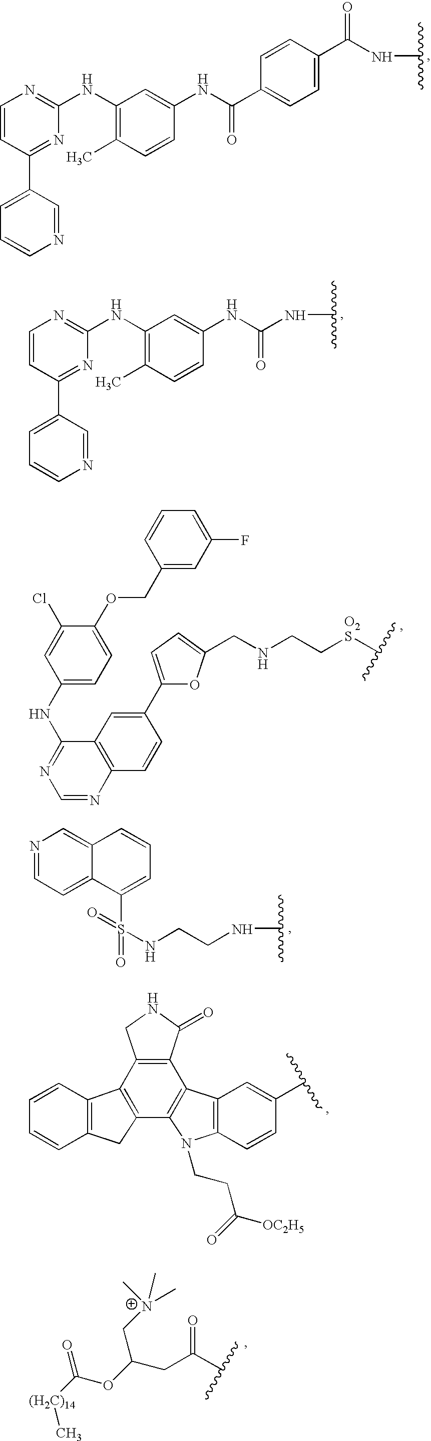

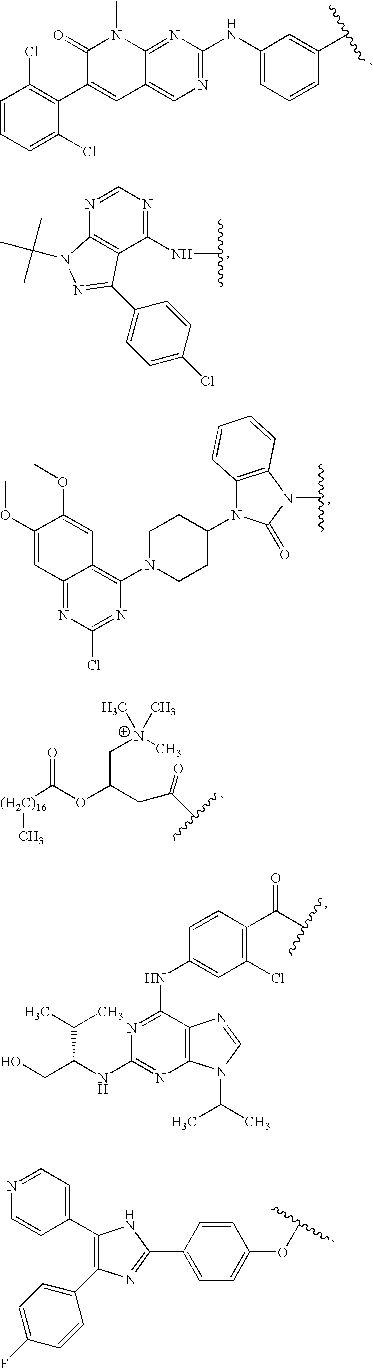

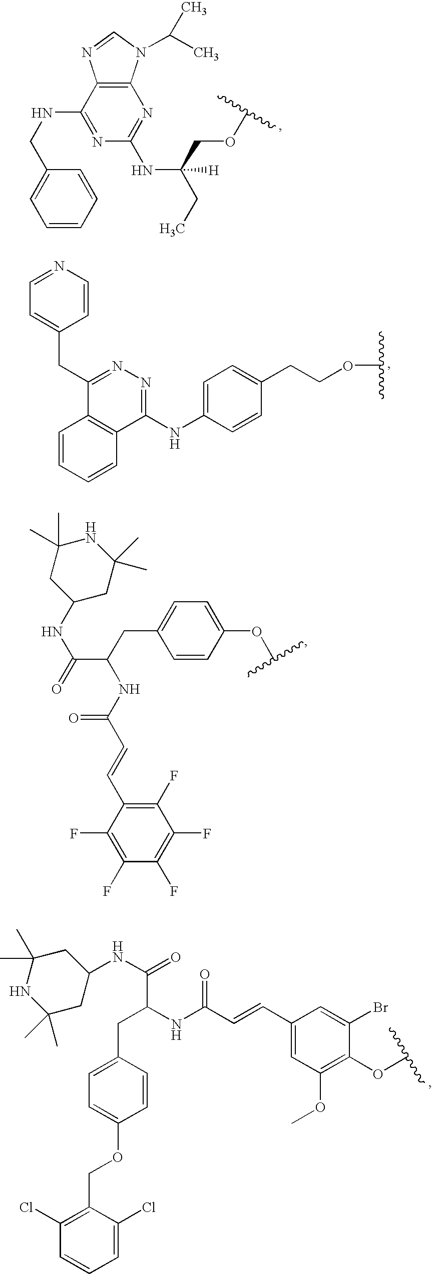

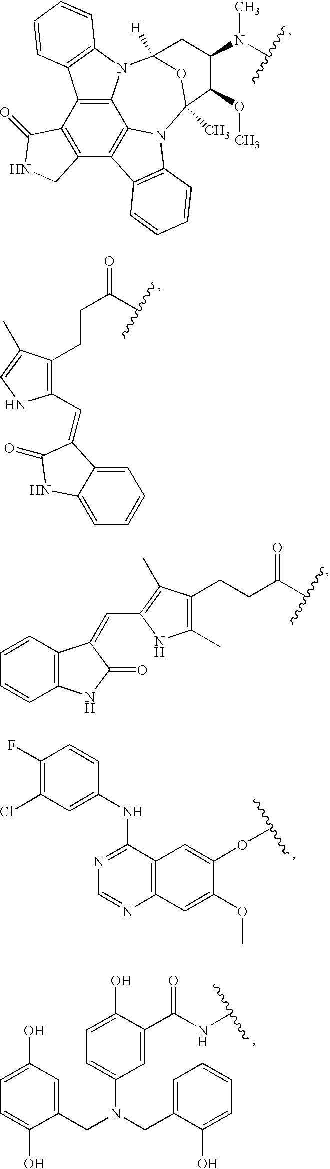

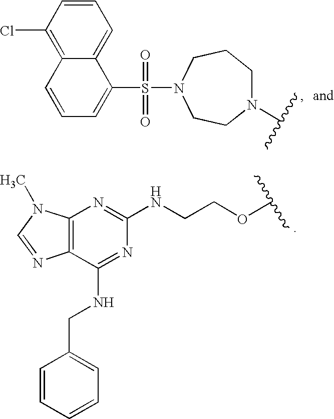

In another or related aspect are compounds selected from the group consisting of compound 27, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, and 74.

In another or related aspect are compounds having the structure:

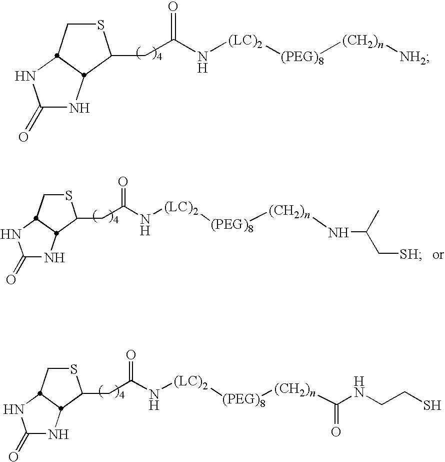

wherein n is 0, 1, or 2, LC is (CH₂)₅C(O)NH, and PEG is (CH₂CH₂O).

In one aspect, the invention provides a linker compound comprising the structure:

X((CR₁R₂)₅—C(═X₁)X₂)ₙ—(CR₃R₄CR₅R₆X₃)ₘ—CR₇R₈CR₉R₁₀X₄

wherein X and X₄ are independently selected functional groups capable of forming a covalent linkage; X₁, X₂, and X₃ are independently selected from the group consisting of O, S, and NH; R₁, R₂, R₃, R₄, R₅, R₆, R₇, R₈, R₉, and R₁₀ are independently selected from the group consisting of hydrogen, halogen, lower alkyl, aryl, and heteroaryl; n is 1, 2, or 3; and m is 6, 7, 8, 9, or 10. X and X₄ can be a carboxylic acid group, an amine group, an aminoxy group, a hydrazide group, a semicarbazide group, a hydroxyl group, a thiol group, an isocyanate group, a thioisocyanate group, a maleimide group, a carboxylic acid derivative, a halide, azide, or a boronic acid derivative.

In another aspect, the invention provides a linker compound comprising the structure:

X((CH₂)₅—C(═O)NH)ₙ—(CH₂CH₂O)ₘ—CR₇R₈CR₉R₁₀X₄

wherein X and X₄ are independently selected functional group capable of forming a covalent linkage; n is 1, 2, or 3; and m is 6, 7, 8, 9, or 10. X and X₄ can be a carboxylic acid group, an amine group, an aminoxy group, a hydrazide group, a semicarbazide group, a hydroxyl group, a thiol group, an isocyanate group, a thioisocyanate group, a maleimide group, and a carboxylic acid derivative.

In another aspect, the invention provides a compound comprising the structure:

A-((CR₁R₂)₅—C(═X₁)X₂)ₙ—(CR₃R₄CR₅R₆X₃)ₘ—CR₇R₈CR₉R₁₀X₄

wherein A is a component useful in biopharmaceutical or bioanalytical applications; X₄ is a functional group capable of forming a covalent linkage; X₁, X₂, and X₃ are independently selected from the group consisting of O, S, and NH; R₁, R₂, R₃, R₄, R₅, R₆, R₇, R₈, R₉, and R₁₀ are independently selected from the group consisting of hydrogen, halogen, lower alkyl, aryl, and heteroaryl; n is 1, 2, or 3; and m is 6, 7, 8, 9, or 10. X₄ can be a carboxylic acid group, an amine group, an aminoxy group, a hydrazide group, a semicarbazide group, a hydroxyl group, a thiol group, an isocyanate group, a thioisocyanate group, a maleimide group, and a carboxylic acid derivative. A can be a binding agent, such as biotin, antigen, antibody, riboflavin, cytostatin, and val-phosphate; a label compound, such as fluorescent label is selected from fluorescein, rhodamine, FITC (fluorescein isothiocyanate), HEX (4,5,2′,4′,5′,7′-hexachloro-6-carboxyfluorescein), 5-IAF, TAMRA (6-carboxytetramethylrhodamine), TET (4,7,2′,7′-tetrachloro-6-carboxyfluorescein), XRITC (rhodamine-X-isothiocyanate), Texas Red®, Cy2, CY3 and CY5; a metal surface binding agent such as thiol or imidazole groups or a therapeutic agent.

In another aspect, the invention provides a compound comprising the structure:

A-Y—((CR₁R₂)₅—C(═X₁)X₂)ₙ—(CR₃R₄CR₅R₆X₃)ₘ—CR₇R₈CR₉R₁₀—B

wherein A and B are independently selected components useful in biopharmaceutical or bioanalytical applications; Y is selected from the group consisting of an amide linkage, an amine linkage, an ether linkage, a thioether linkage, an ester linkage, a thioester linkage, a urea linkage, a thiourea linkage, a carbamate linkage, a thiocarbamate linkage, a Schiff base linkage, a reduced Schiff base linkage, an oxime linkage, a semicarbazide linkage, a hydrazone linkage and a carbon-carbon linkage; X₁, X₂, and X₃ are independently selected from the group consisting of O, S, and NH; R₁, R₂, R₃, R₄, R₅, R₆, R₇, R₈, R₉, and R₁₀ are independently selected from the group consisting of hydrogen, halogen, lower alkyl, aryl, and heteroaryl; n is 1, 2, or 3; and m is 6, 7, 8, 9, or 10. A and B can be independently selected to be a binding agent, such as biotin, antigen, antibody, riboflavin, cytostatin, and val-phosphate; a label compound, such as fluorescent label is selected from fluorescein, rhodamine, FITC (fluorescein isothiocyanate), HEX (4,5,2′,4′,5′,7′-hexachloro-6-carboxyfluorescein), 5-IAF, TAMRA (6-carboxytetramethylrhodamine), TET (4,7,2′,7′-tetrachloro-6-carboxyfluorescein), XRITC (rhodamine-X-isothiocyanate), Texas Red®, Cy2, CY3 and CY5; or a therapeutic agent such as spiramycin, ipriflavone, mesalazine, and crotamiton.

In another aspect, the invention provides new and novel compounds 27, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, and 74.

In one aspect, the invention provides a compound comprising the structure:

Z-(CR₃R₄CR₅R₆X₃)ₘ—CR₇R₈CR₉R₁₀X₄—B

wherein Z is H or R₁₁ where R₁₁ is selected from the group consisting of lower alkyl, aryl, heteroaryl, X((CR₁R₂)₅—C(═X₁)X₂)ₙ—, A-((CR₁R₂)₅—C(═X₁)X₂)ₙ—, and A-Y—((CR₁R₂)₅—C(═X₁)X₂)ₙ—; X and X₄ are independently selected functional groups capable of forming a covalent linkage or is a direct bond; X₁, X₂, and X₃ are independently selected from the group consisting of O, S, and NH; R₁, R₂, R₃, R₄, R₅, R₆, R₇, R₈, R₉, and R₁₀ are independently selected from the group consisting of hydrogen, halogen, lower alkyl, aryl, and heteroaryl; n is 1, 2, or 3; m is 6, 7, 8, 9, or 10; T is a direct bond or a triazole; A is a component useful in biopharmaceutical or bioanalytical applications; Y is selected from the group consisting of an amide linkage, an amine linkage, an ether linkage, a thioether linkage, an ester linkage, a thioester linkage, a urea linkage, a thiourea linkage, a carbamate linkage, a hydrazide linkage, a thiocarbarnate linkage, a Schiff base linkage, a reduced Schiff base linkage, an oxime linkage, a semicarbazide linkage, a hydrazone linkage and a carbon-carbon linkage; and B is a therapeutic agent. In some embodiments, the therapeutic agent, B, is a kinase inhibitor, such as one or more of the compounds I, II, III, IV, V, VI, VII, VII, IX, X, XI, XII, XIII, XIV, XV, XVI, XVII, XVIII, IX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXI or XL as described herein.

In another aspect, compounds comprising the structure Z′-X₄—B are provided where B is a kinase inhibitor; X₄ is a direct bond or a linking group having from 1 to 3 atoms independently selected from unsubstituted or substituted carbon, N, O or S; Z′ is selected from the group consisting of H, lower alkyl, aryl, heteroaryl, X((CR₁R₂)₅—C(═X₁)X₂)ₙ-T-(CR₃R₄CR₅R₆X₃)ₘ—CR₇R₈CR₉R₁₀—, A-((CR₁R₂)₅—C(X₁)X₂)ₙ-T-(CR₃R₄CR₅R₆X₃)ₘ—CR₇R₈CR₉R₁₀—, A-Y—((CR₁R₂)₅—C(═X₁)X₂)ₙ-T-(CR₃R₄CR₅R₆X₃)ₘ—CR₇R₈CR₉R₁₀—, and X((CR₁R₂)₅—C(═X₁)X₂)ₙ-T-(CR₃R₄CR₅R₆X₃)ₘ—CR₇R₈CR₉R₁₀—, wherein X is a functional group capable of forming a covalent linkage or is a direct bond; X₁, X₂, and X₃ are independently selected from the group consisting of O, S, and NH; R₁, R₂, R₃, R₄, R₅, R₆, R₇, R₈, R₉, and R₁₀ are independently selected from the group consisting of hydrogen, halogen, lower alkyl, aryl, and heteroaryl; n is 1, 2, or 3; m is 6, 7, 8, 9, or 10; T is a direct bond or a triazole; A is a component useful in biopharmaceutical or bioanalytical applications; and Y is selected from the group consisting of an amide linkage, an amine linkage, an ether linkage, a thioether linkage, an ester linkage, a thioester linkage, a urea linkage, a thiourea linkage, a carbamate linkage, a hydrazide linkage, a thiocarbamate linkage, a Schiff base linkage, a reduced Schiff base linkage, an oxime linkage, a semicarbazide linkage, a hydrazone linkage and a carbon-carbon linkage.

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (1992) “ADVANCED ORGANIC CHEMISTRY 3^{RD} ED.” Vols. A and B, Plenum Press, New York. Unless otherwise indicated, conventional methods of mass spectroscopy, NMR, HPLC, protein chemistry, biochemistry, recombinant DNA techniques and pharmacology, within the skill of the art are employed.

The term “agonist” means a molecule such as a compound, a drug, an enzyme activator or a hormone that enhances the activity of another molecule or the activity of a receptor site.

The term “alkenyl group” includes a monovalent unbranched or branched hydrocarbon chain having one or more double bonds therein. The double bond of an alkenyl group can be unconjugated or conjugated to another unsaturated group. Suitable alkenyl groups include, but are not limited to, (C₂-C₈)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted.

The term “alkoxy” as used herein includes —O-(alkyl), wherein alkyl is defined above.

The term “lower alkyl” means a straight chain or branched, saturated or unsaturated chain having from 1 to 10 carbon atoms. Representative saturated alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, and n-hexyl, and longer alkyl groups, such as heptyl, and octyl. An alkyl group can be unsubstituted or substituted. Unsaturated alkyl groups include alkenyl groups and alkynyl groups, discussed below. Alkyl groups containing three or more carbon atoms may be straight, branched or cyclized.

The term “alkynyl group” includes a monovalent unbranched or branched hydrocarbon chain having one or more triple bonds therein. The triple bond of an alkynyl group can be unconjugated or conjugated to another unsaturated group. Suitable alkynyl groups include, but are not limited to, (C₂-C₆)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl group can be unsubstituted or substituted.

The term “aryl” includes a carbocyclic or heterocyclic aromatic group containing from 5 to 30 ring atoms. The ring atoms of a carbocyclic aromatic group are all carbon atoms, and include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. A carbocyclic aromatic group can be unsubstituted or substituted. Preferably, the carbocyclic aromatic group is a phenyl group. The ring atoms of a heterocyclic aromatic group contains at least one heteroatom, preferably 1 to 3 heteroatoms, independently selected from nitrogen, oxygen, and sulfur. Illustrative examples of heterocyclic aromatic groups include, but are not limited to, pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidinyl, tetrazolyl, furyl, thienyl, isoxazolyl, thiazolyl, furyl, phienyl, isoxazolyl, indolyl, oxetanyl, azepinyl, piperazinyl, morpholinyl, dioxanyl, thietanyl and oxazolyl. A heterocyclic aromatic group can be unsubstituted or substituted. Preferably, a heterocyclic aromatic is a monocyclic ring, wherein the ring comprises 2 to 5 carbon atoms and 1 to 3 heteroatoms.

The term “aryloxy” includes —O-aryl group, wherein aryl is as defined above. An aryloxy group can be unsubstituted or substituted.

The term “cycloalkyl” includes a monocyclic or polycyclic saturated ring comprising carbon and hydrogen atoms and having no carbon-carbon multiple bonds. Examples of cycloalkyl groups include, but are not limited to, (C₃-C₇)cycloalkyl groups, such as cyclopropyl, cyclobutyl, cyclopentyl, cyclohexyl, and cycloheptyl, and saturated cyclic and bicyclic terpenes. A cycloalkyl group can be unsubstituted or substituted. Preferably, the cycloalkyl group is a monocyclic ring or bicyclic ring.

The terms “effective amount” or “therapeutically effective amount” refer to a sufficient amount of the agent to provide the desired biological result. That result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined by one of ordinary skill in the art using routine experimentation.

The term“halogen” includes fluorine, chlorine, bromine, and iodine. By “pharmaceutically acceptable” or “pharmacologically acceptable” is meant a material which is not biologically or otherwise undesirable, i.e., the material may be administered to an individual without causing undesirable biological effects or interacting in a deleterious manner with any of the components of the composition in which it is contained.

The term “pharmaceutically acceptable salt” of a compound means a salt that is pharmaceutically acceptable and that possesses the desired pharmacological activity of the parent compound. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the present invention, the mammal is a human.

The term “sulfonyl” refers to the presence of a sulfur atom, which is optionally linked to another moiety such as an aliphatic group, an aromatic group, an aryl group, an alicyclie group, or a heterocyclic group. Aryl or alkyl sulfonyl moieties have the formula —SO₂R′, and alkoxy moieties have the formula —O—R′, wherein R′ is alkyl, as defined above, or is aryl wherein aryl is phenyl, optionally substituted with 1-3 substituents independently selected from halo (fluoro, chloro, bromo or iodo), lower alkyl (1-6C) and lower alkoxy (1-6C).

The terms “treating” and its grammatical equivalents as used herein includes achieving a therapeutic benefit and/or a prophylactic benefit. By therapeutic benefit is meant eradication, amelioration, or prevention of the underlying disorder being treated or the eradication, amelioration, or prevention of one or more of the physiological symptoms associated with the underlying disorder such that an improvement is observed in the patient, notwithstanding that the patient may still be afflicted with the underlying disorder. For a prophylactic benefit, for example, the compositions described herein maybe administered to a patient at risk of developing a particular disease or to a patient reporting one or more of the physiological symptoms of that disease, even though a diagnosis of the disease may not have been made.

Unless otherwise indicated, when a substituent is deemed to be “optionally substituted,” it is meant that the substituent is a group that may be substituted with one or more group(s) individually and independently selected from, for example, alkyl, cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, perhaloalkyl, perfluoroalkyl, silyl, trihalomethanesulfonyl, and amino, including mono- and di-substituted amino groups, and the protected derivatives thereof. The protecting groups that may form the protective derivatives of the above substituents are known to those of skill in the art.

Molecular embodiments of the present invention may possess one or more chiral centers and each center may exist in the R or S configuration. The present invention includes all diastereomeric, enantiomeric, and epimeric forms as well as the appropriate mixtures thereof. Stereoisomers may be obtained, if desired, by methods known in the art as, for example, the separation of stereoisomers by chiral chromatographic columns. Additionally, the compounds of the present invention may exist as geometric isomers. The present invention includes all cis, trans, syn, anti, entgegen (E), and zusammen (Z) isomers as well as the appropriate mixtures thereof. In some situations, compounds may exist as tautomers. All tautomers are included within the formulas described herein are provided by this invention.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

These and other aspects of the present invention will become evident upon reference to the following detailed description. In addition, various references are set forth herein which describe in more detail certain procedures or compositions, and are incorporated by reference in their entirety.

### BRIEF DESCRIPTION OF THE FIGURES

FIG. 1 illustrates the NMR spectra of 6-(6-tert-Butoxycarbonylamino-hexanoylamino)-hexanoic acid N-{2-[2-Azidoethoxy-octakis(2-ethoxy)]ethyl}amide (compound 17).

FIG. 2 illustrates the NMR spectra of 6-[5-(2-Oxo-hexahydro-thieno[3,4-d]imidazol-4-yl)-pentanoylamino]-hexanoic acid [5-{2-(2-amino-ethoxy)-octakis(2-ethoxy)}-ethylcarbamoyl)-pentyl]-amide (compound 22).

### DETAILED DESCRIPTION OF THE INVENTION

### The Compounds

The present invention is generally directed to a linker compound useful for forming a linkage between a conjugate of multiple components. Preferably the linkage formed is a stable linkage. In the case where the conjugate comprises two components, the linker compound forms a linkage, preferably a stable linkage, between a first component and a second component. In some embodiments, the present invention discloses linker compounds that are stable and that provide the means to control and determine the distance between the first component and the second component. Further, the linker compounds of the present invention can be selected such that the conjugate is either more or less hydrophobic. In some embodiments, the linker compounds comprise of polyethyleneglycol (PEG) and aminocaproic acid (“LC” for Long Chain) that are capable of forming linkages with a carboxyl-, aldehyde-, amine-, hydroxy-, thiol-, or arylhalide-containing component. The number and order of repeating units of PEG and LC that comprise the linker compounds can be selected such that the length between the first component and the second component, as well as the hydrophobic and hydrophilic characteristics of the linker can be controlled.

Accordingly, in one aspect, the present invention provides linked conjugates comprising a first component covalently linked to a second component through a linkage facilitated by a linker compound. Such conjugates can be generally represented by structure I:

A-linker-B (I)

where A is a first component and B is a second component, and the linker is covalently joined to both A and B. Preferably, the linked conjugates are linked through a stable linkage.

In some embodiments, the linked conjugates comprise a first component covalently linked to a second component through a linkage formed by a linker compound which comprises a hydrophobic unit and a hydrophilic unit, such as depicted below:

A-Hydrophobic unit-Hydrophilic unit-B

In a preferred embodiment, the hydrophobic unit comprise of one or more aminocaproic acid units and the hydrophilic unit comprises of 4 or more PEG units.

In one aspect, the invention provides linker compounds comprising the structure:

X((CR₁R₂)ₚ—C(═X₁)X₂)ₙ—(CR₃R₄CR₅R₆X₃)ₘ—CR₇R₈CR₉R₁₀X₄

wherein X and X₄ are independently selected functional groups capable of forming a covalent linkage; X₁, X₂, and X₃ are independently selected from the group consisting of O, S, and NH; R₁, R₂, R₃, R₄, R₅, R₆, R₇, R₈, R₉, and R₁₀ are independently selected from the group consisting of hydrogen, halogen, alkyl, aryl, and heteroaryl; p is equal to or greater than 1; n is equal to or greater than 1; and m is equal to or greater than 3. X and X₄ can be a carboxylic acid group, an amine group, an aminoxy group, a hydrazide group, a semicarbazide group, a hydroxyl group, a thiol group, an isocyanate group, a thioisocyanate group, a maleimide group, a halide, azide, a boronic acid derivative or a carboxylic acid derivative. For example, in some embodiments p ranges from 1 to 10. In other embodiments, p ranges from 2 to 8. In still other embodiments, p is equal to 5. In some embodiments, n ranges from 1 to 3. In some embodiments, m ranges from 4 to 20. In other embodiments, m ranges from 7 to 11.

In another aspect, the invention provides linker compounds comprising the structure:

X((CH₂)ₚ—C(═O)NH)ₙ—(CH₂CH₂O)ₘ—CR₇CR₉R₁₀X₄

wherein X and X₄ are independently selected functional group capable of forming a covalent linkage; R₇, R₈, R₉, and R₁₀ are independently selected from the group consisting of hydrogen, halogen, alkyl, aryl, and heteroaryl; p is equal to or greater than 1; n is equal to or greater than 1; and m is equal to or greater than 3. X and X₄ can be a carboxylic acid group, an amine group, an aminoxy group, a hydrazide group, a semicarbazide group, a hydroxyl group, a thiol group, an isocyanate group, a thioisocyanate group, a maleimide group, a halide, azide, a boronic acid derivative or a carboxylic acid derivative. In some embodiments, p ranges from 1 to 10. In other embodiments, p ranges from 2 to 8. In still other embodiments, p is equal to 5. In some embodiments, n ranges from 1 to 3. In some embodiments, m ranges from 4 to 20. In other embodiments, m ranges from 7 to 11.

In another aspect, the invention provides compounds comprising the structure:

A-((C₁R₂)ₚ—C(═X₁)X₂)ₙ—(CR₃R₄CR₅R₆X₃)ₘ—CR₇R₈CR₉R₁₀X₄

wherein A is a component useful in biopharmaceutical or bioanalytical applications; X₄ is a functional group capable of forming a covalent linkage; X₁, X₂, and X₃ are independently selected from the group consisting of O, S, and NH; R₁, R₂, R₃, R₄, R₅, R₆, R₇, R₈, R₉, and R₁₀ are independently selected from the group consisting of hydrogen, halogen, alkyl, aryl, and heteroaryl; p is equal to or greater than 1; n is equal to or greater than 1; and m is equal to or greater than 3. X₄ can be a carboxylic acid group, an amine group, an aminoxy group, a hydrazide group, a semicarbazide group, a hydroxyl group, a thiol group, an isocyanate group, a thioisocyanate group, a maleimide group, a halide, azide, a boronic acid derivative or a carboxylic acid derivative. A can be a binding agent, a label compound, or a therapeutic agent. In some embodiments p ranges from 1 to 10. In other embodiments, p ranges from 2 to 8. In still other embodiments, p is equal to 5. In some embodiments, n ranges from 1 to 3. In some embodiments, m ranges from 4 to 20. In other embodiments, m ranges from 7 to 11.

In another aspect, the invention provides compounds comprising the structure:

A-Y—((CR₁R₂)ₚ—C(═X₁)X₂)ₙ—(CR₃R₄CR₅R₆X₃)ₘ—CR₇R₈CR₉R₁₀—B

wherein A and B are independently selected components useful in biopharmaceutical or bioanalytical applications; Y is selected from the group consisting of an amide linkage, an amine linkage, an ether linkage, a thioether linkage, an ester linkage, a thioester linkage, a urea linkage, a thiourea linkage, a hydrazide linkage, a carbamate linkage, a thiocarbamate linkage, a Schiff base linkage, a reduced Schiff base linkage, an oxime linkage, a semicarbazide linkage, a hydrazone linkage and a carbon-carbon linkage; X₁, X₂, and X₃ are independently selected from the group consisting of O, S, and NH; R₁, R₂, R₃, R₄, R₅, R₆, R₇, R₈, R₉, and R₁₀ are independently selected from the group consisting of hydrogen, halogen, alkyl, aryl, and heteroaryl; p is equal to or greater than 1; n is equal to or greater than 1; and m is equal to or greater than 3. A and B can be independently selected to be a binding agent, a label compound, or a therapeutic agent. In some embodiments, p ranges from 1 to 10. In other embodiments, p ranges from 2 to 8. In still other embodiments, p is equal to 5. In some embodiments, n ranges from 1 to 3. In some embodiments, m ranges from 4 to 20. In other embodiments, m ranges from 7 to 11.

In one aspect, the invention provides a compound comprising the structure:

Z—(CR₃R₄CR₅R₆X₃)ₘ—CR₇R₈CR₈R₁₀X₄—B

wherein Z is H or R₁₁ where R₁₁ is selected from the group consisting of lower alkyl, aryl, heteroaryl, X((CR₁R₂)ₚ—C(═X₁)X₂)ₙ—, A-((CR₁R₂)ₚ—C(═X₁)X₂)ₙ—, and A-Y—((CR₁R₂)ₚ—C(═X₁)X₂)ₙ—; X and X₄ are independently selected functional groups capable of forming a covalent linkage or is a direct bond; X₁, X₂, and X₃ are independently selected from the group consisting of O, S, and NH; R₁, R₂, R₃, R₄, R₅, R₆, R₇, R₈, R₉, and R₁₀ are independently selected from the group consisting of hydrogen, halogen, lower alkyl, aryl, and heteroaryl; p is equal to or greater than 1; n is equal to or greater than 1; and m is equal to or greater than 3; A is a component useful in biopharmaceutical or bioanalytical applications; Y is selected from the group consisting of an amide linkage, an amine linkage, an ether linkage, a thioether linkage, an ester linkage, a thioester linkage, a urea linkage, a thiourea linkage, a carbamate linkage, a hydrazide linkage, a thiocarbamate linkage, a Schiff base linkage, a reduced Schiff base linkage, an oxime linkage, a semicarbazide linkage, a hydrazone linkage and a carbon-carbon linkage; and B is a therapeutic agent. In some embodiments, the therapeutic agent, B, is a kinase inhibitor, such as one or more of the compounds I, II, III, IV, V, VI, VII, VIII, IX, X, XI, XII, XIII, XIV, XV, XVI, XVII, XVIII, IX, XX, XXI, XXI, XXIII, XXIV, XXV, XXVI, XXVII, XXVIII, XXIX, XXX, XXXI, XXXII, XIII, XXXIV, XXXV, XXXVI, XXXVII, XXXVIII, XXXIX or XL as disclosed herein. In some embodiments, p ranges from 1 to 10. In other embodiments, p ranges from 2 to 8. In still other embodiments, p is equal to 5. In some embodiments, n ranges from 1 to 3. In some embodiments, m ranges from 4 to 20. In other embodiments, m ranges from 7 to 11. Examples of other kinase inhibitors that can be conjugated with the linker compounds described herein are known in the art.

In another aspect, compounds comprising the structure Z′—X₄—B are provided where B is a kinase inhibitor; X₄ is a direct bond or a linking group having from 1 to 3 atoms independently selected from unsubstituted or substituted carbon, N, O or S; Z′ is selected from the group consisting of H, lower alkyl, aryl, heteroaryl, X((CR₁R₂)ₚ—C(═X₁)X₂)ₙ-T-(CR₃R₄CR₅R₆X₃)ₘ—CR₇R₈CR₉R₁₀—, A-((CR₁R₂)ₚ—C(═X₁)X₂)ₙ-T-(CR₃R₄CR₅R₆X₃)ₘ—CR₇R₈CR₉R₁₀—, A-Y—((CR₁R₂)ₚ—C(═X₁)X₂)ₙ-T-(CR₃R₄CR₅R₆X₃)ₘ—CR₇R₈CR₉R₁₀—, and X((CR₁R₂)ₚ—C(═X₁)X₂)ₙ-T-(CR₃R₄CR₅R₆X₃)ₘ—CR₇R₈CR₉R₁₀—, wherein X is a functional group capable of forming a covalent linkage or is a direct bond; X₁, X₂, and X₃ are independently selected from the group consisting of O, S, and NH; R₁, R₂, R₃, R₄, R₅, R₆, R₇, R₈, R₉, and R₁₀ are independently selected from the group consisting of hydrogen, halogen, lower alkyl, aryl, and heteroaryl; p is equal to or greater than 1; n is equal to or greater than 1; and m is equal to or greater than 3; T is a direct bond or a triazole; A is a component useful in biopharmaceutical or bioanalytical applications; and Y is selected from the group consisting of an amide linkage, an amine linkage, an ether linkage, a thioether linkage, an ester linkage, a thioester linkage, a urea linkage, a thiourea linkage, a carbamate linkage, a hydrazide linkage, a thiocarbamate linkage, a Schiff base linkage, a reduced Schiff base linkage, an oxime linkage, a semicarbazide linkage, a hydrazone linkage and a carbon-carbon linkage. In some embodiments, p ranges from 1 to 10. In other embodiments, p ranges from 2 to 8. In yet other embodiments, p is equal to 5. In some embodiments, n ranges from 1 to 3. In some embodiments, m ranges from 4 to 20. In other embodiments, m ranges from 7 to 11.

In another aspect, the invention provides new and novel compounds 27, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, and 74.

In one aspect of this invention, the A component can be a binding agent that is capable of binding to a specific binding partner. A wide variety of binding agents can be utilized, such as antibodies and antibody fragments which recognize a selected antigen, and by further screening of such antibodies in order to select those with a high affinity, riboflavin that binds to riboflavin binding protein, cytostatin that binds to papain with an affinity of 10⁻¹⁴ M, val-phosphonate that binds to carboxypeptidase A with an affinity of 10⁻¹⁴ M, 4CABP that binds to RuBisCo with an affinity of 10⁻¹³ M, and biotin that binds to avidin having an affinity of 10⁻¹⁵ M. In some embodiments, the A component is biotin, which is readily detectable by of its binding to avidin or streptavidin. For immunoassay and immunocytochemistry applications, avidin or streptavidin may themselves be labeled, either directly or indirectly, or may be bound to a solid support. Examples of immunoassays employing biotin-labeled (biotinylated) ligands and avidin or streptavidin are given in the following references: U.S. Pat. Nos. 4,863,876, 5,028,524, and 5,371,516. Nucleic acid hybridization assays can also be performed using a biotinylated probe to visualize a specific sequence of interest. Hybridization assays employing biotinylated probes and avidin or streptavidin are given in Yamane et al, Nuc. Acids Symp. Ser. 21:9, 1989, and Baretton et al., Cancer Res. 54:4472, 1994. Immunoaffinity chromatography employing biotinylated antibodies and immobilized avidin is described in Hofman et al., J. Am. Chem. Soc. 100:3585, 1978, Bayer et al., Meth. Enzymol. 62:308, 1979, and U.S. Pat. Nos. 5,225,353, 5,215,927, and 5,262,334.

In addition to binding agents, the A component may be a label compound that reports the presence of the linker compound or linked conjugate to which the label is attached. Examples of suitable labels include fluorescent labels, enzymes, enzyme substrates, and radioactive labels. The labels can be detected spectroscopically and include fluorescent, phosphorescent, luminescent, and chromagenic molecules. The fluorescent labels include fluorescein, rhodamine, FITC (fluorescein isothiocyanate), HEX (4,5,2′,4′,5′,7′-hexachloro-6-carboxyfluorescein), 5-IAF, TAMRA (6-carboxytetramethylrhodamine), TET (4,7,2′,7′-tetrachloro-6-carboxyfluorescein), XRITC (rhodamine-X-isothiocyanate), Texas Red®, Cy2, CY3, CY5 and other cyanine derivatives as well as fluorescent proteins such as phycobiliproteins. As label compounds, enzymes and enzyme substrates generate detectable signals upon enzymatic action. The use of enzymes as labels is well known. Common enzymes for labeling purposes include, for example, alkaline phosphatase, horseradish peroxidase, β-galactosidase, and luciferase. Typical enzyme substrate labels include chemiluminescent compounds such as dioxetanes which emit light upon enzymatic action.

Radioactive labels include compounds that bear radioactive isotopes, for example, radioisotopes of hydrogen, carbon, sulfur, phosphorous, as well as radioactive metals such as Cu-64, Ga-67, Ga-68, Zr-89, Ru-97, Tc-99m, Rh-105, Pd-109, In-111, I-123, I-125, I-131, Re-186, Au-198, Au-199, Pb-203, At-211, Pb-212 and Bi-212.

As mentioned above, the A component possesses a functional group to effect covalent coupling to the linker compound. For example, where the A component is biotin, biotin may be directly coupled to the linker compound through biotin's carboxylic acid group. In such a coupling, the covalent linkage between biotin and the linker compound may be accomplished by amide bond formation (where X in the structures above is amine). Alternatively, the A component may contain additional functional groups. For example, where the first component is biotin, commercially available reactive derivatives of biotin contain groups which effectively increase the distance between the biotin moiety and the reactive terminus of the biotin derivative. These biotin derivatives extend the biotin reactive coupling site by the addition of, for example, diamine or amino acid moieties to biotin's carboxylic acid group. Like biotin, the biotin amino acid derivative presents a carboxylic acid functional group for coupling to the linker compound. In contrast, the biotin diamine derivative presents an amino group for coupling to the linker compound, and thus the X moiety in the linkers of the invention may be a carboxylic acid group, an amine group, an aminoxy group, a hydrazide group, a semicarbazide group, a hydroxyl group, a thiol group, an isocyanate group, a thioisocyanate group, a maleimide group, or a carboxylic acid derivative.

In the practice of the present invention, any covalent linkage, preferably a stable covalent linkage, may be employed to join the A component with the linker compound. For example, when the A component is a label compound such as fluorescein, the label may contain functional groups such as isothiocyanate (—N═C═S), or a reactive ester such as an N-hydroxysuccinimide ester to form the covalent linkage between the A component to the linker compound. The fluorescein derivatives containing the isothiocyanate and the N-hydroxysuccinimide ester are commercially available from a variety of sources, and covalent linkage to the linker compound may be accomplished through thiourea or amide bond formation, respectively.

The linker compounds of the present invention serve to form the linkages between a first component (the A component) to a second component (the B component) to provide a linked conjugate. Preferably, the linkages formed are stable linkages and the resulting conjugates are stably-linked conjugates. In the practice of the present invention, the B component can be any molecule or compound identified above with regard to the A component, and which contains (or is modified to contain) a suitably reactive carbonyl moiety, such as an aldehyde or ketone. As with the A component, numerous molecules and compounds are known and may be utilized in this regard.

Thus, the B component can be the same as the A component, and can be a binding agent, a label compound, or a therapeutic agent. The therapeutic agent can be selected for detecting, preventing and treating conditions associated with ischemic cell death, such as myocardial infarction, stroke, glaucoma, and other neurodegenerative conditions.

The therapeutic agent can be selected such that it has activity against a variety of diseases and unwanted conditions, including, but not limited to, cerebral accident (or cerebrovascular accident, including stroke), inflammation (including inflammation due to autoimmune diseases), multiple sclerosis, blood vessel growth (angiogenesis), bone formation/bone growth, immune system stimulation, acute coronary syndromes (including myocardial infarction, non-Q-wave myocardial infarction and unstable angina), cardiovascular disease, arthritis (including osteoarthritis, degenerative joint disease, spondyloarthropathies, gouty arthritis, systemic lupus erythematosus, juvenile arthritis and rheumatoid arthritis), common cold, dysmenorrhea, menstrual cramps, inflammatory bowel disease, Crohn's disease, emphysema, acute respiratory distress syndrome, asthma, bronchitis, chronic obstructive pulmonary disease, Alzheimer's disease, organ transplant toxicity, cachexia, allergic reactions, allergic contact hypersensitivity, cancer (such as solid tumor cancer including colon cancer, breast cancer, lung cancer and prostrate cancer; hematopoietic malignancies including leukemias and lymphomas; Hodgkin's disease; aplastic anemia, skin cancer and familiar adenomatous polyposis), tissue ulceration, peptic ulcers, gastritis, regional enteritis, ulcerative colitis, diverticulitis, recurrent gastrointestinal lesion, gastrointestinal bleeding, coagulation, anemia, synovitis, gout, ankylosing spondylitis, restenosis, periodontal disease, epidermolysis bullosa, osteoporosis, atherosclerosis (including atherosclerotic plaque rupture), aortic aneurysm (including abdominal aortic aneurysm and brain aortic aneurysm), periarteritis nodosa, congestive heart failure, myocardial infarction, stroke, cerebral ischemia, head trauma, spinal cord injury, neuralgia, neurodegenerative disorders (acute and chronic), autoimmune disorders, Huntington's disease, Parkinson's disease, migraine, depression, peripheral neuropathy, pain (including low back and neck pain, headache and toothache), gingivitis, cerebral amyloid angiopathy, nootropic or cognition enhancement, amyotrophic lateral sclerosis, multiple sclerosis, ocular angiogenesis, corneal injury, macular degeneration, conjunctivitis, abnormal wound healing, muscle or joint sprains or strains, tendonitis, skin disorders (such as psoriasis, eczema, scleroderma and dermatitis), myasthenia gravis, polymyositis, myositis, bursitis, burns, diabetes (including types I and II diabetes, diabetic retinopathy, neuropathy and nephropathy), tumor invasion, tumor growth, tumor metastasis, corneal scarring, scleritis, immunodeficiency diseases (such as AIDS in humans and FLV, FIV in cats), sepsis, premature labor, hypoprothrombinemia, hemophilia, thyroiditis, sarcoidosis, Behcet's syndrome, hypersensitivity, kidney disease, Rickettsial infections (such as Lyme disease, Erlichiosis), Protozoan diseases (such as malaria, giardia, coccidia), reproductive disorders (preferably in livestock) and septic shock (preferably arthritis, fever, common cold, pain and cancer).

Thus, for example, biotin-LC-LC-(PEG)₈-CH₂CH₂—NH₂, can be linked to the antibacterial compound spiramycin I, as shown below.

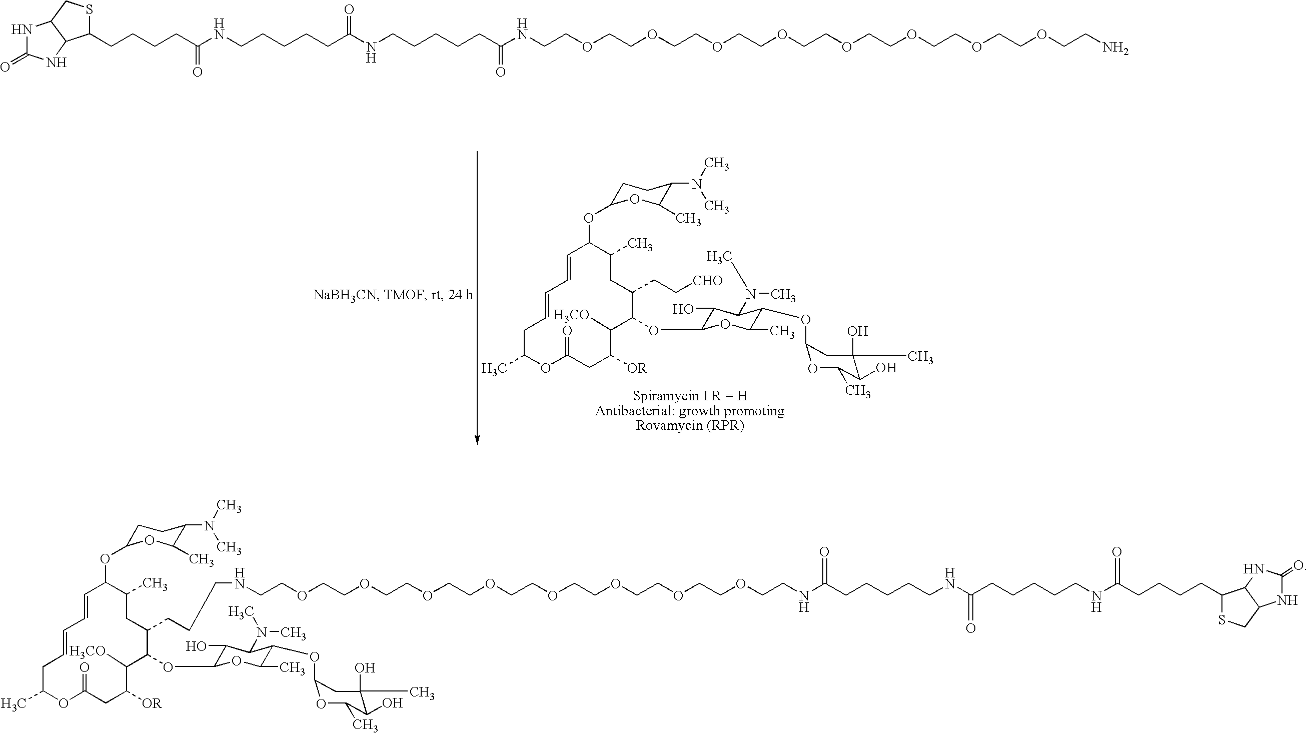

In another example, a calcium regulator can he linked to biotin using the linkers of the invention as illustrated below.

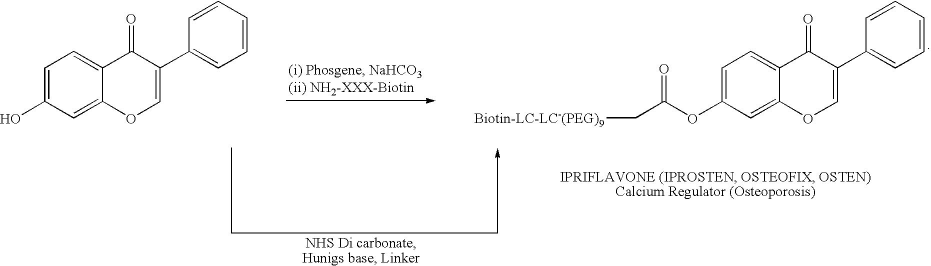

In another example, a drug for gastrointestinal disorders can be linked to biotin using the linkers of the invention to provide the conjugate illustrated below.

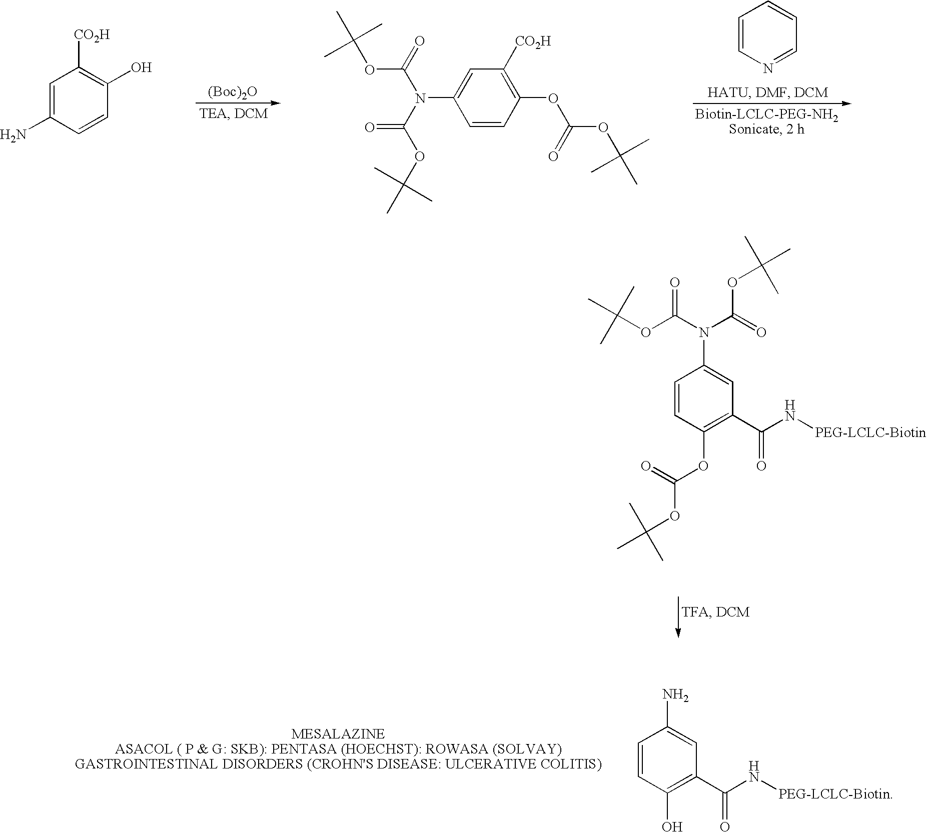

In yet another example, an antipruritic can be linked to the linkers of the invention to provide the conjugate as illustrated below.

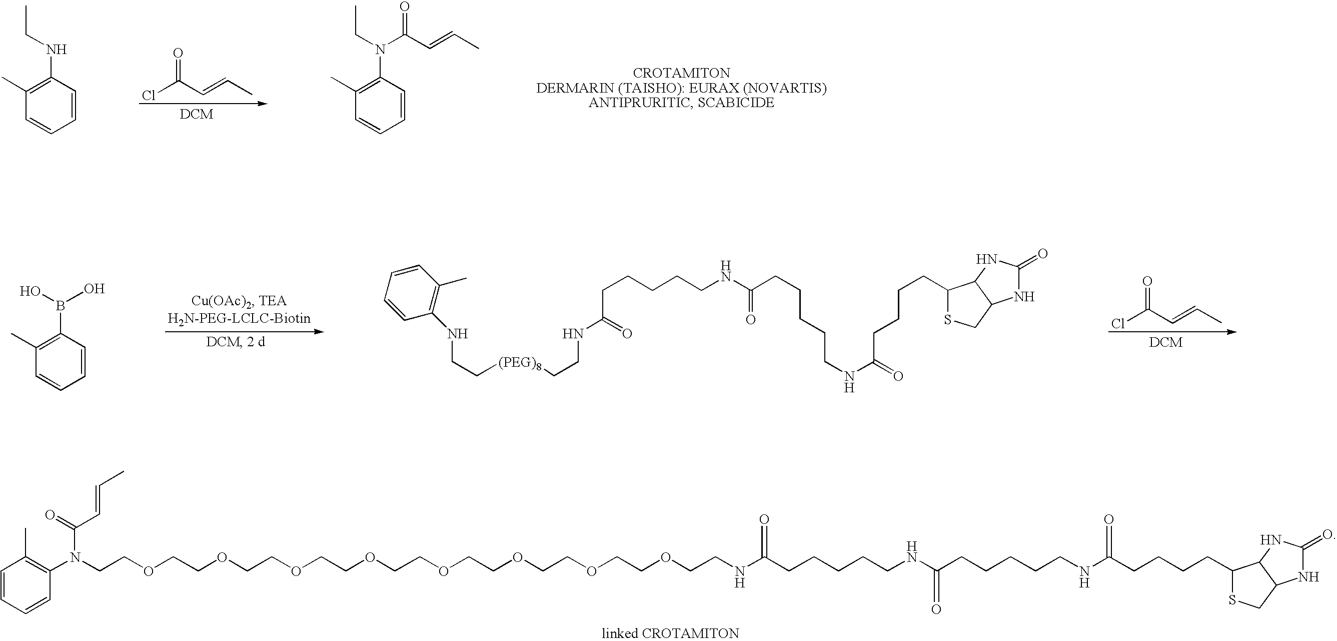

In yet another example, the therapeutic agent can be a kinase inhibitor. The kinase inhibitor can be any kinase inhibitor, such as one or more of the following compounds:

| Compound | |
|---|---|
| No. | Structure |
| I | 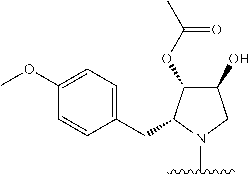 |
| II | 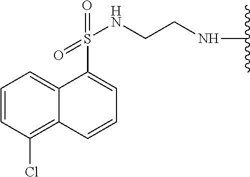 |
| III | 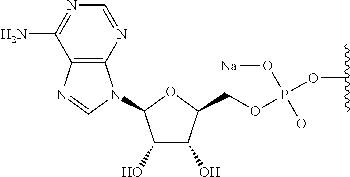 |
| IV | 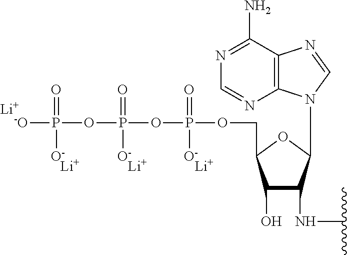 |
| V | 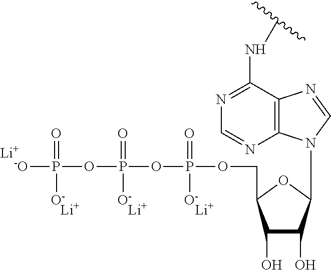 |
| VI | 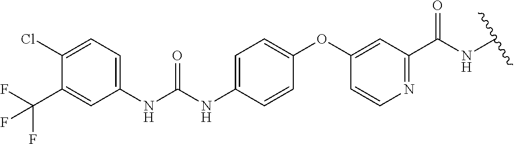 |
| VII | 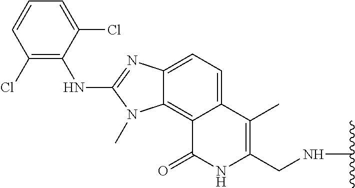 |
| VIII | 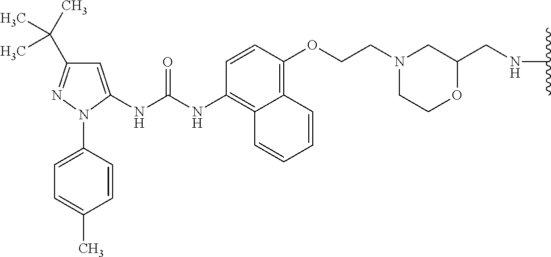 |
| IX | 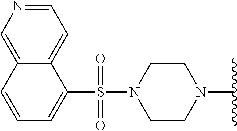 |
| X | 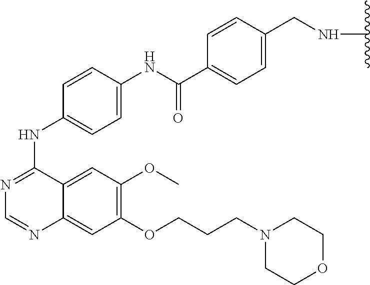 |
| XI | 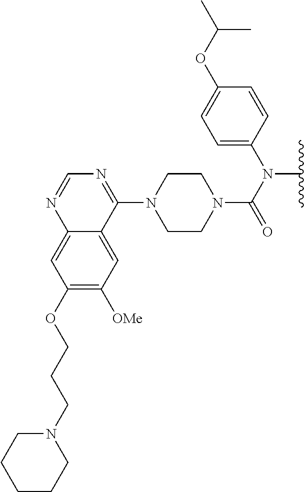 |
| XII | 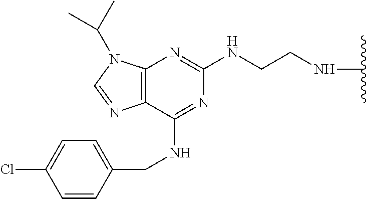 |
| XIII | 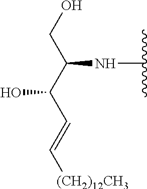 |
| XIV | 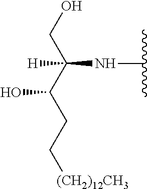 |
| XV | 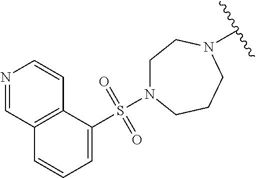 |
| XVI | 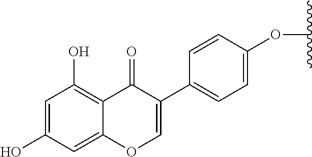 |
| XVII | 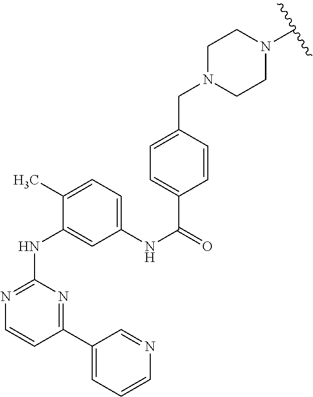 |
| XVIII | 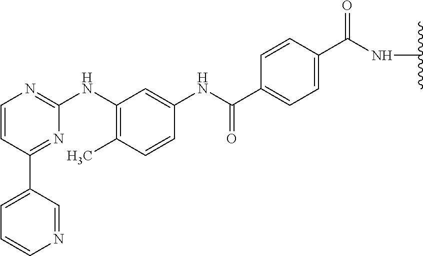 |
| IX (XIX) | 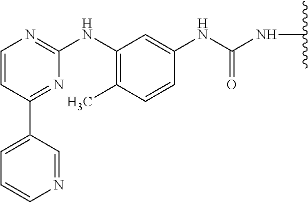 |
| XX | 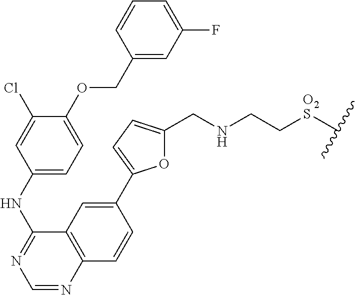 |
| XXI | 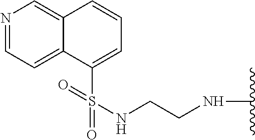 |
| XXII | 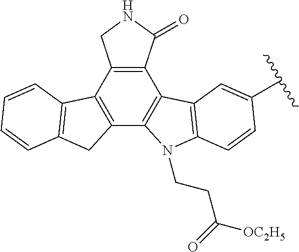 |
| XXIII | 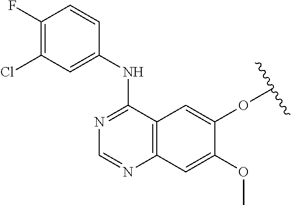 |
| XXIV | 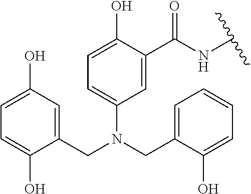 |
| XXV | 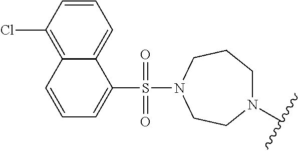 |
| XXVI | 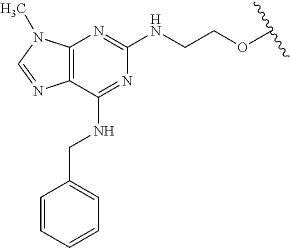 |
| XXVII | 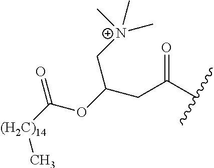 |
| XXVIII | 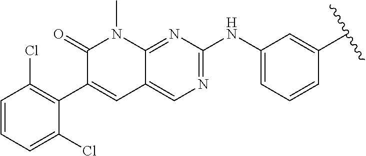 |
| XXIX | 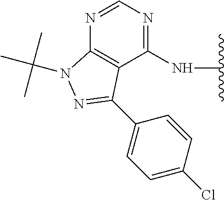 |
| XXX | 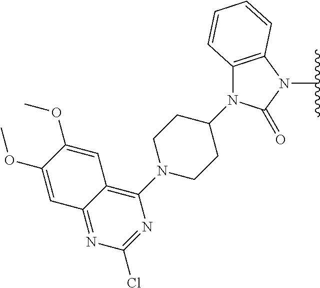 |
| XXXI | 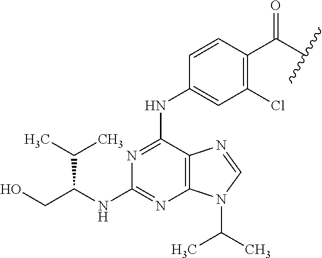 |
| XXXII | 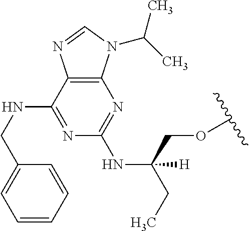 |
| XXXIII | 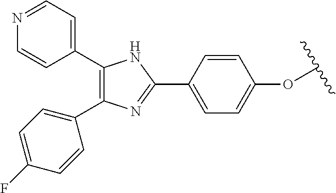 |
| XXXIV | 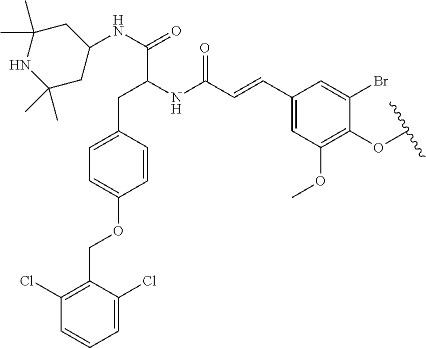 |
| XXXV | 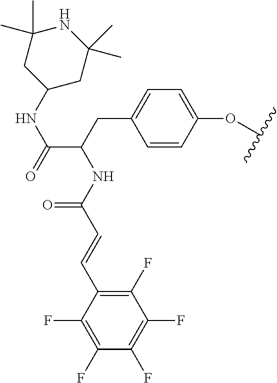 |
| XXXVI | 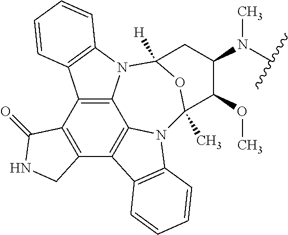 |
| XXXVII | 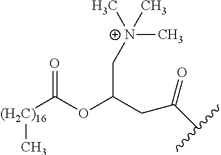 |
| XXXVIII | 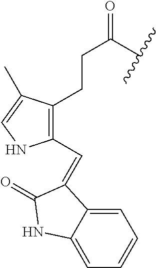 |
| XXXIX | 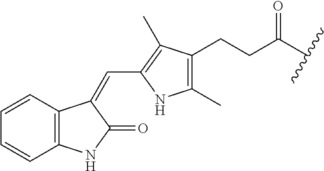 |
| XL | 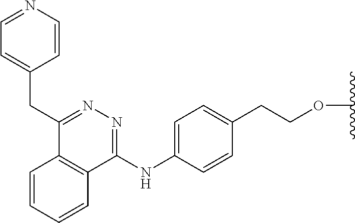 |

In one aspect, the kinase inhibitor can be modified to contain functional groups capable of forming a covalent linkage. For example, the kinase inhibitors can be attached to X₄ where X₄ can be a carboxylic acid group, an amine group, an aminoxy group, a hydrazide group, a semicarbazide group, a hydroxyl group, a thiol group, an isocyanate group, a thioisocyanate group, a maleimide group, a halide, azide, a boronic acid derivative or a carboxylic acid derivative.

In another aspect, the kinase inhibitor can be covalently attached to polyethyleneglycol (PEG) and aminocaproic acid (LC). The kinase inhibitor can be attached to PEG or LC either directly or via the functional group X₄ described in detail above.

As will be evident to one of skill in the art, the terminal amino group can be reacted with various thiol group to give a terminal thiol group, as illustrated below.

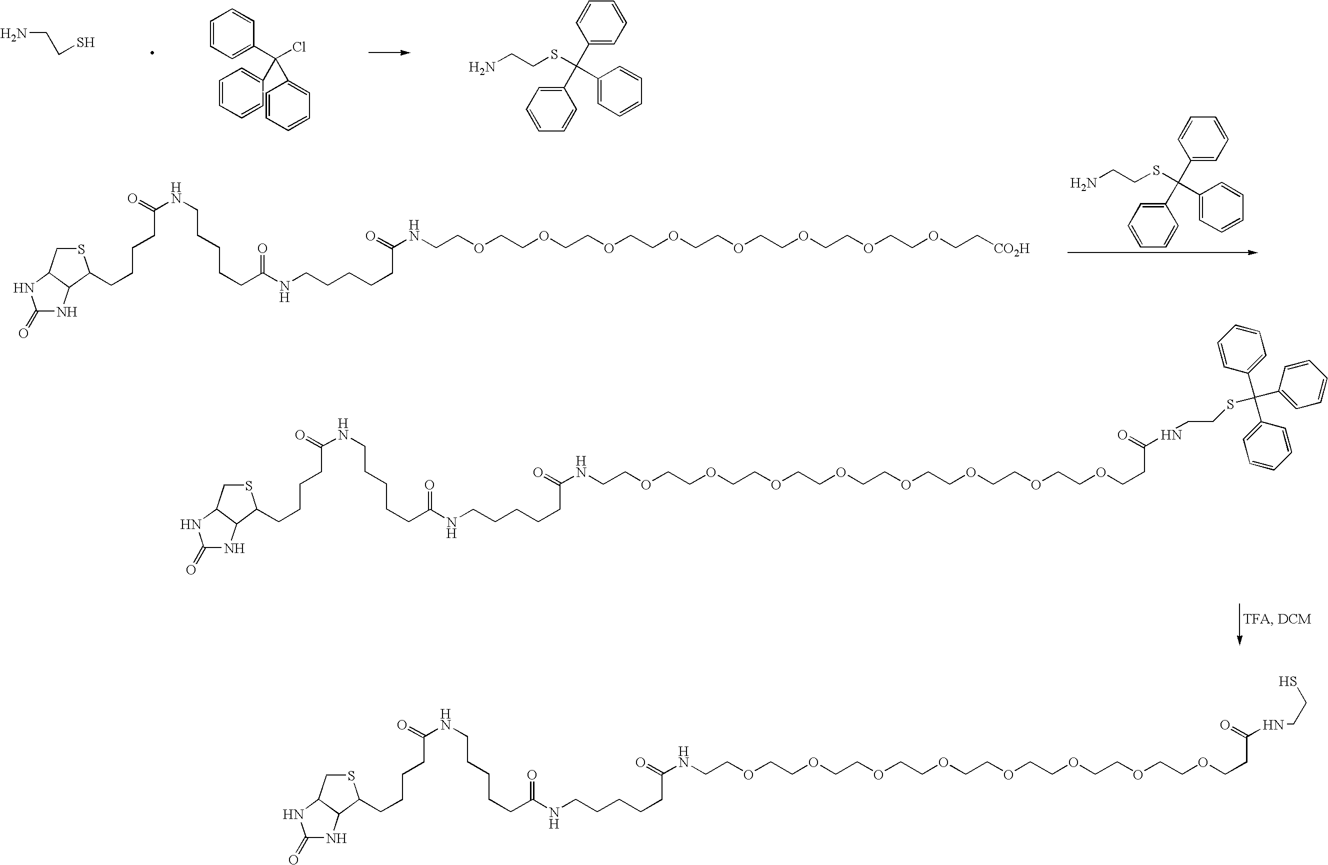

In another aspect of the invention, the PEG and the LC portions of the linker can be connected using a triazole ring. In one aspect, the triazole ring can have substituents that can be used to covalently link PEG and LC units. In another aspect, the triazole ring is created, such as by metal catalyzed coupling of an azido group and an alkyne group, as illustrated in the general synthetic scheme below:

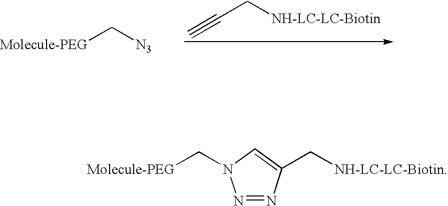

Thus, in one aspect, the invention discloses compounds of the type:

X((CR₁R₂)₅—C(═X₁)X₂)ₙ—(CH₂)ₙ-T-(CR₃R₄CR₅R₆X₃)ₘ—CR₇R₈CR₉R₁₀X₄

wherein T is the triazole, and the other substituents are as described above. The use of the triazole ring to link the PEG and LC portions together can be advantageous when the conversion of the azido group to the amine group prior to coupling is not preferred, especially if aryl halides are present in the linked molecule, which could be hydrogenated off under Pd/H conditions.

### Methods of Preparation

The linkers, conjugates, and other compounds of the invention can be synthesized as described in detail in the Examples. The compounds of the present invention and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTY 3^{rd} Ed., Vols. A and B (Plenum 1992), and Green and Wuts, PROTECTIVE GROUPS IN ORGANIC SYNTHESIS 2^{nd} Ed. (Wiley 1991). Starting materials for the compounds of the invention may be obtained using standard techniques and commercially available precursor materials, such as those available from Aldrich Chemical Co., Sigma Chemical Co, Lancaster Synthesis (Windham, N.H.), Apin Chemicals, Ltd. (New Brunswick, N.J.), Ryan Scientific (Columbia, S.C.), Molecular Biosciences (Boulder, Colo.) and Maybridge. Starting materials useful for preparing compounds of the invention and intermediates thereof are commercially available or can be prepared by well-known synthetic methods (see, e.g., Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-21, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” 3d Edition, John Wiley & Sons, 1995).

The procedures described herein for synthesizing the compounds of the invention may include one or more steps of protection and deprotection (e.g., the formation and removal of acetal groups). Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (e.g., methyl and ethyl esters, acetate or propionate groups or glycol esters) or alkylated such as benzyl and trityl ethers, as well as alkyl ethers, tetrahydropyranyl ethers, trialkylsilyl ethers (e.g., TMS or TIPPS groups) and allyl ethers.

In addition, the synthetic procedures disclosed below can include various purifications, such as column chromatography, flash chromatography, thin-layer chromatography (TLC), recrystallization, distillation, high-pressure liquid chromatography (HPLC) and the like. Also, various techniques well know in the chemical arts for the identification and quantification of chemical reaction products, such as proton and carbon-13 nuclear magnetic resonance (¹H and ¹³C NMR), infrared and ultraviolet spectroscopy (IR and UV), X-ray crystallography, elemental analysis (EA), HPLC and mass spectroscopy (MS) can be used as well. Methods of protection and deprotection, purification and identification and quantification are well known in the chemical arts.

An example of a procedure for linking primary alcohols is illustrated in the synthetic scheme below:

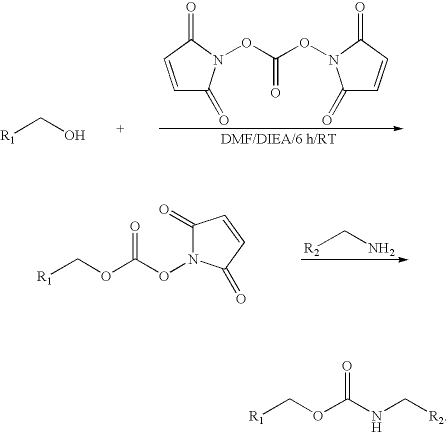

Typically, one equivalent of the alcohol, 1.2 equivalents of N,N-disuccinimidyl carbonate and five equivalents of DIEA can be dissolved in dry DMF (1M solution) and the mixture can be stirred at room temperature for approximately six hours. One equivalent of the amino linker can then be added and the reaction mixture can be stirred at room temperature for 24 hours. The final product can be purified by HPLC. This procedure can be used for linking primary alcohols in the presence of secondary alcohols.

For example, corticosterone can be linked to biotin using the linker (PEG)ₙ-LC-LC as illustrated in the synthetic scheme below:

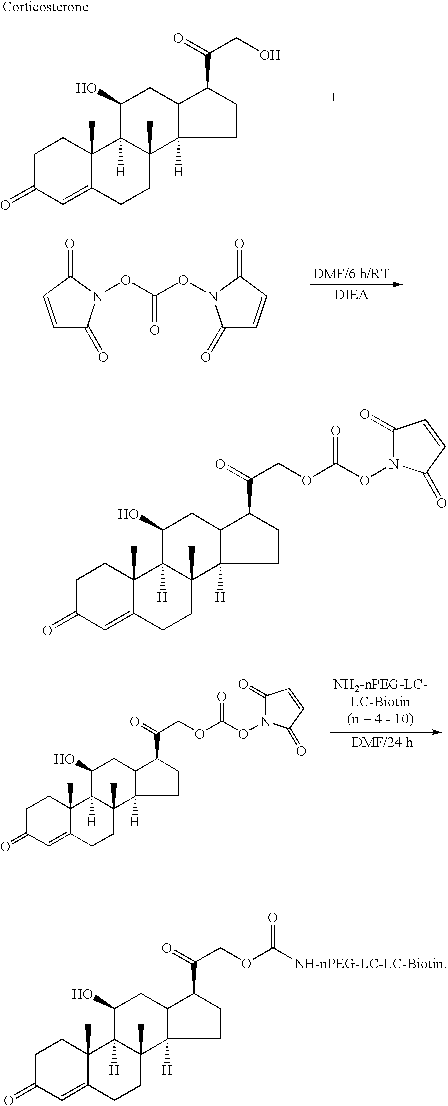

Approximately 25 mg (1 eq.) of corticosterone, 22 mg (1.2 eq.) of the linker and 0.1 ml (5 eq.) of DIEA can be dissolved in 2 mL of dry DMF. The reaction mixture can be stirred at room temperature for about 6 h then 1 eq. of the linker can be added. After stirring at room temperature for 24 h, the final product can be purified by HPLC to give a yield of about 68% to 90% of the product.

A general procedure for linking phenols is illustrated in the synthetic scheme below:

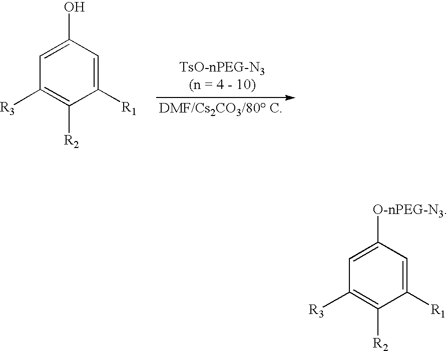

Typically, one equivalent of the phenol, 1.2 equivalents of the TsO-nPEG-N₃ and 1.2 equivalents of cesium carbonate can be suspended in dry DMF and the reaction mixture can be stirred at 80° C. for 16 h. The organic solvent can be evaporated in vacuum and the resulting residue can be dissolved in ethyl acetate. The ethyl acetate can be then washed with water, dried with magnesium sulfate and evaporated to dryness. The final product can be purified by preparative HPLC.

In one procedure, illustrated below, the PEGylated compound can than be dissolved in methanol (1M solution) and 10 mol % of 20% Pd/C can be added. The reaction mixture can be stirred under hydrogen for approximately 12 h, typically followed by filtration through a Celite 545 pad. The resulting solution can be evaporated in vacuum and the primary amine coupled with 1.1 eq NHS-LC-LC-Biotin in presence of HATU/DIEA in DMF:

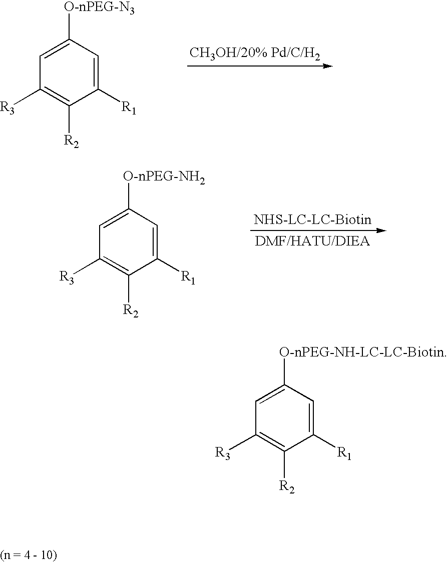

In another procedure, useful for functional groups sensitive to palladium mediated hydrogenation, the reduction of the azide to an amine can be carried out by using Staudinger reduction. A typical procedure for Staudinger reduction is illustrated in the synthetic scheme below:

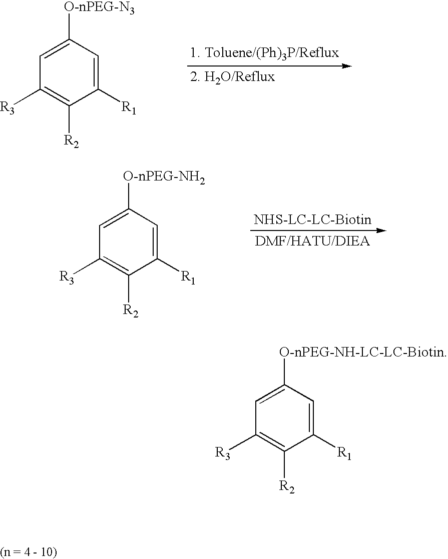

Typically, 1 eq. of the azide and 1.5 eq of triphenylphosphine can be dissolved in dry toluene and refluxed under nitrogen for 24 h. Ten eq. of water can be added and the reflux can be continued for another 6 h. The resulting primary amine can be purified by preparative HPLC and coupled with NHS-LC-LC-Biotin in presence of HATU/DIEA in DMF.

For example, estradiol can be linked to biotin selectively at the phenolic functional group over the unprotected secondary alcohol using the procedure below:

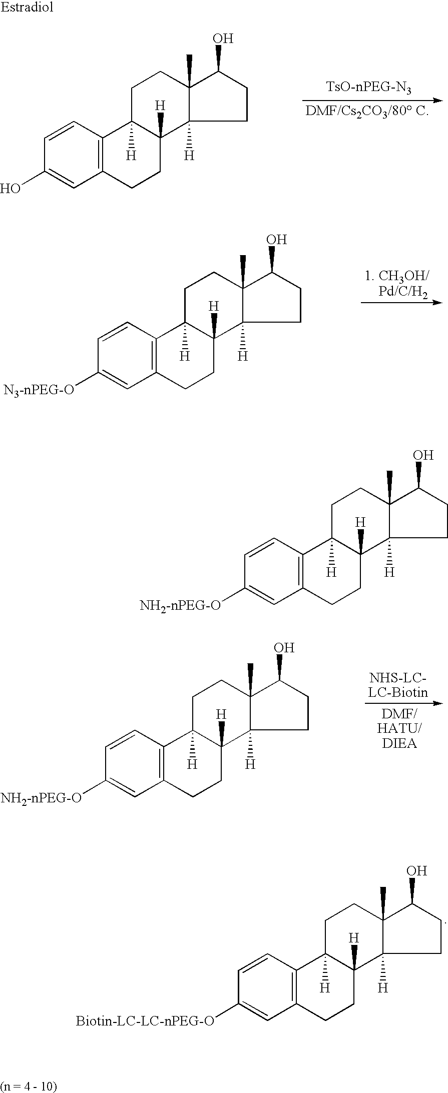

Thus, 1 molar eq. estradiol, 1.1 molar eq. of TsO-nPEG-N₃ and 1.2 molar eq. Cs₂CO₃ can be suspended in 2 ml of dry DMF and stirred at about 80° C. for approximately 16 h. The organic solvent can be evaporated in vacuum and the residue suspended in 10 ml ethyl acetate, washed with water (3×30 ml), dried over magnesium sulfate, and evaporated to dryness. The final product can be purified by preparative HPLC.

The pegylated estradiol can than be dissolved in methanol (1M solution) and 10 mol % of 20% Pd/C can be added. The reaction mixture can be stirred under hydrogen for 12 h and then filtered through a Celite 545 pad. The resulting solution can be evaporated in vacuum and the primary amine coupled with (1.1 eq) of NHS-LC-LC-Biotin in the presence of (2 eq.) HATU, 0.1 ml DIEA in 3 ml of dry DMF to give the final product.

Similarly, in one embodiment, the phenolic group can be linked selectively over the tertiary alcohol in ethinylestradiol as illustrated below:

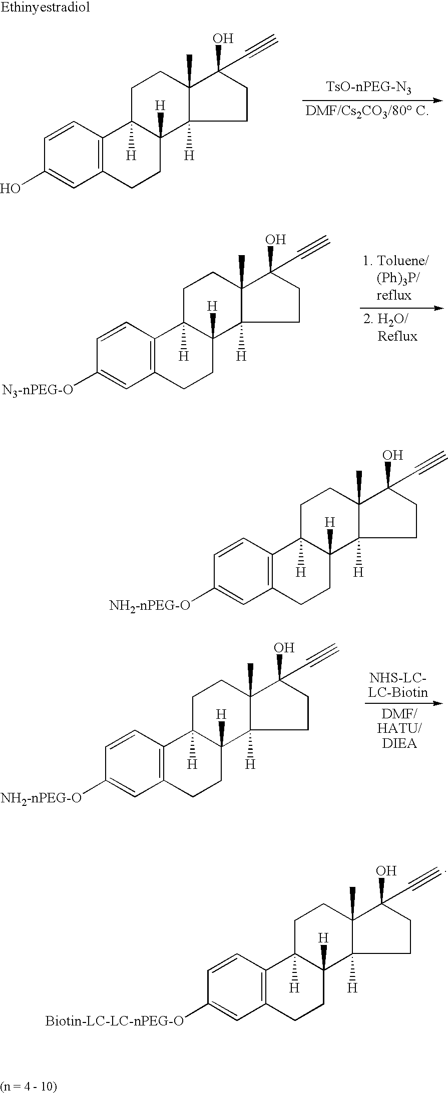

One molar eq. ethinylestradiol, 1.1 molar eq. of TsO-nPEG-N₃, and 1.2 molar eq. Cs₂CO₃ can be suspended in dry DMF and stirred at about 80° C. for about 16 h. The organic solvent can be evaporated in vacuum and the residue suspended in ethyl acetate, washed three times with water, dried over magnesium sulfate and evaporated to dryness. The final product can be purified by preparative HPLC.

In the next step, 1 molar eq. of the azide and 1.5 molar eq. of triphenylphosphine can be dissolved in dry toluene and refluxed under nitrogen for about 24 h. Then, 10 eq. of water can be added and the reflux continued for another 6 h. The final product can be purified by HPLC.

Finally, 1 molar eq. of the primary amine can be coupled with 1.1 molar eq. of NHS-LC-LC-Biotin in presence of 2 molar eq. of HATU, 0.1 ml DIEA in dry DMF to give the linked ethinylestradiol.

In another embodiment, metal catalysis can be used to couple an azido group and an alkyne group, as illustrated in the general synthetic scheme below:

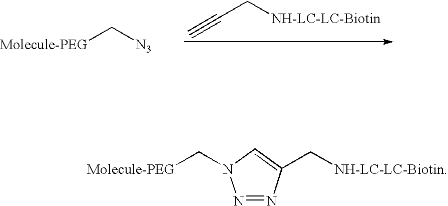

In the first step, the alkyne-functionalized LC-LC-biotin can be prepared using commercially available reagents:

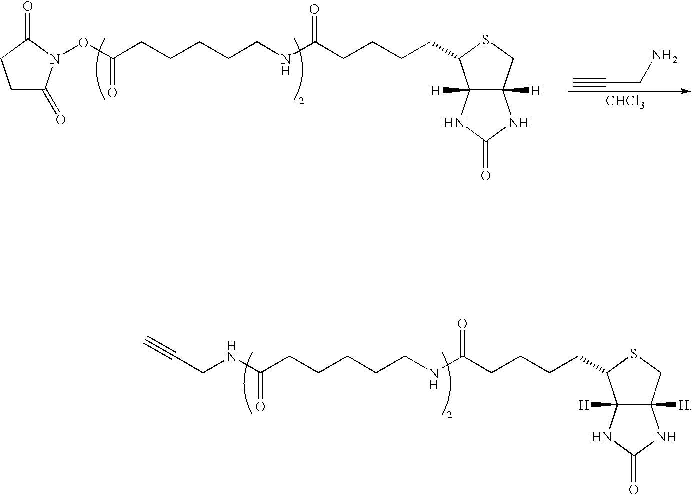

Then, the azido-PEG functionalized compound can be coupled to form the biotin-conjugate:

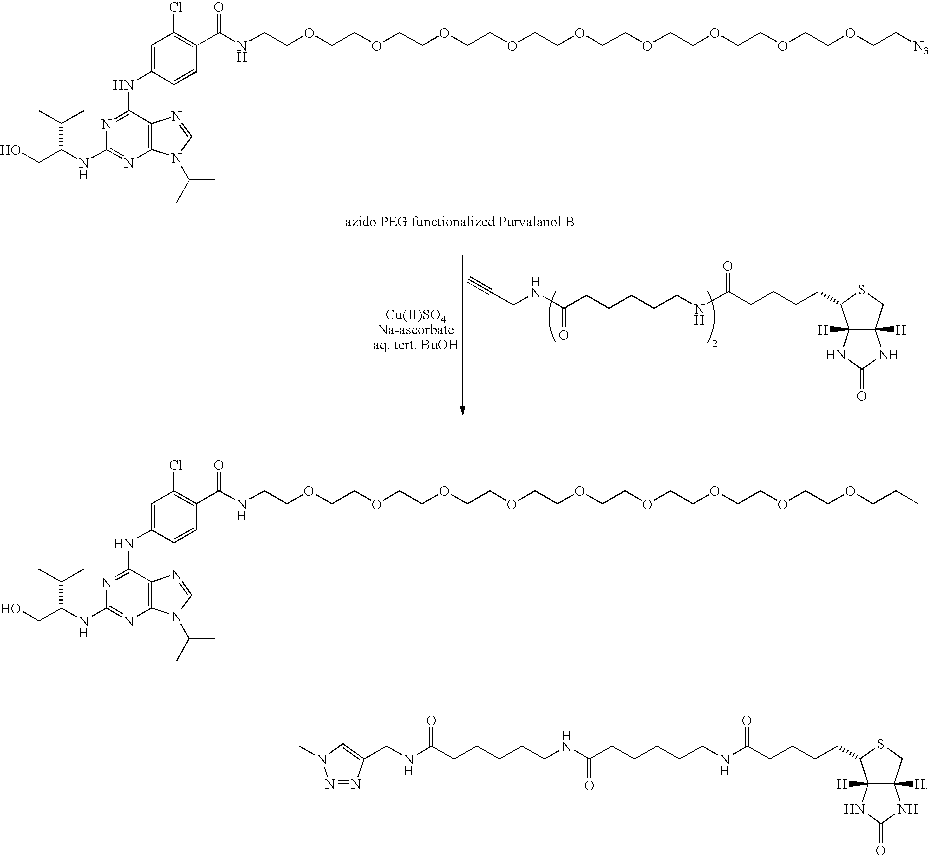

### Methods of Use: Screening Assays and Diagnostic Applications

The linkers and linked conjugates described herein can be used to detect a variety of biological components such as antigens, haptens, monoclonal and polyclonal antibodies, gene probes, natural and synthetic oligo- and polynucleotides, natural and synthetic mono- oligo- and polysaccharides, growth factors, hormones, receptor molecules, as well as mixtures thereof. Also, the compounds described herein can be used to detect various micro-organisms, such as bacteria, viruses, fungi, prions, etc.

The linked conjugates of this invention are useful, for example, for a variety of diagnostic and separation techniques. There are a variety of assay formats, e.g., immunoassays, known to those of ordinary skill in the art for using a conjugate as described herein to detect diagnostic molecules (e.g., antigens, proteins, peptides, and other bio-molecules that indicate the presence of a disease or infection) in a sample. In one embodiment, the A component can be a binding agent capable of binding to a specific binding partner and the B component can be an antibody that recognizes the molecule of interest. The assay can then be performed by incubating the stably-linked conjugate with the sample, for a period of time sufficient to permit binding of the antibody to the antigen, and then separating the conjugate-antigen complex from the remainder of the sample. Such separation can be achieved by, for example, contacting the sample with an immobilized compound capable of binding to the conjugate-antigen complex. For example, if the A component is biotin, a solid support containing immobilized avidin or streptavidin can be used to remove conjugate-antigen complex from the sample. Bound complex can then be detected using a second binding partner (e.g., Protein A or an antibody that binds to the conjugate-antigen complex). The solid support can be any solid material known to those of ordinary skill in the art to which the antigen can be attached. For example, the solid support can be a test well in a microtiter plate or a nitrocellulose or other suitable membrane. Alternatively, the support can be a bead or disc, such as glass, fiberglass, latex or a plastic material such as polystyrene or polyvinylchloride.

In another embodiment, the immunoassay is a two-antibody sandwich assay. This assay can be performed by first contacting an antibody that has been immobilized on a solid support, commonly the well of a microtiter plate or a membrane, with the sample, such that antigen within the sample is allowed to bind to the immobilized antibody. Unbound sample is then removed from the immobilized antigen-antibody complexes and a linked conjugate is added, wherein the A component is a label compound (e.g., an enzyme (such as horseradish peroxidase), substrate, cofactor, inhibitor, dye, radionuclide, luminescent group, or fluorescent group) and the B component is a second antibody capable of binding to a different site on the antigen. The amount of linked conjugate that remains bound to the solid support is then determined using a method appropriate for the specific label compound.

Once the antibody is immobilized on the support as described above, the remaining protein binding sites on the support are typically blocked with a suitable blocking agent. The immobilized antibody is then incubated with the sample and antigen within the sample is allowed to bind to the antibody. Preferably, the incubation time is sufficient to achieve a level of binding that is at least 95% of that achieved at equilibrium between bound and unbound antigen. Those of ordinary skill in the art will recognize that the time necessary to achieve equilibrium can be readily determined by assaying the level of binding that occurs over a period of time. At room temperature, an incubation time of about 30 minutes is generally sufficient.

Unbound sample can then be removed by washing the solid support with an appropriate buffer, and the linked conjugate can be added to the solid support. The linked conjugate is then incubated with the immobilized antibody-antigen complex for an amount of time sufficient to detect the bound antigen. An appropriate amount of time can generally be determined by assaying the level of binding that occurs over a period of time. Unbound linked conjugate is then removed and bound linked conjugate is detected using the label compound. The method employed for detecting the label compound depends upon the nature of the label compound. For radioactive groups, scintillation counting or autoradiographic methods are generally appropriate. Spectroscopic methods can be used to detect fluorescent groups. Enzyme label compounds can generally be detected by the addition of substrate (generally for a specific period of time), followed by spectroscopic, or other analysis of the reaction products.

Linked conjugates can also be used for the separation of a specific cell type from a biological sample. For example, a linked conjugate can be employed in which the A component is a binding agent, such as biotin, and the B component is an antibody or other molecule specific for a cell surface antigen of the desired cell type. Such a linked conjugate can be incubated with an appropriate biological sample and allowed to bind to the surface antigen. The cell-conjugate complex can then be separated from the remainder of the sample by, for example, contacting the sample with an immobilized compound capable of binding to the cell-conjugate complex. For example, if the A component is biotin, a solid support containing immobilized avidin or streptavidin can be used to remove cell-conjugate complex from the sample. Unbound sample constituents can then be removed by an appropriate wash, and the cell separated from the solid support. Representative cell separation procedures can be found in U.S. Pat. Nos. 5,215,927, 5,225,353, and 5,262,334, and published PCT applications WO 92/07243 and WO 92/08988, and the co-owned and co-pending application U.S. serial no. 2003/0186221.

In addition to the in vitro uses mentioned above, the linked conjugates of the present invention also have utility for in vivo diagnostic and therapeutic applications. For example, a typical in vivo use would include in vivo imaging, as well as targeted delivery of therapeutic agents.

The present invention also provides kits for carrying out various assays, diagnostic, and therapeutic techniques. Such kits typically comprise of the linkers and/or linked conjugates described herein. The kits may include additional components useful for carrying out the assays and methods described herein. Examples of additional components include, but are not limited to, labels, buffers, reagents, etc. The kits may also include instructions teaching methods of use of the components of the kit.

### Methods of Use: Therapeutic Uses

The present invention also provides pharmaceutical compositions for treatment of various diseases comprising the linkers or linked conjugates, described herein, as an active ingredient in combination with one or more pharmaceutically suitable carrier. The pharmaceutical compositions of the present invention may further comprise other therapeutically active ingredients. Also provided herein are methods of treating various diseases in a subject suffering therefrom comprising administering to the subject an effective amount of the linkers or linked conjugates, disclosed hereinabove, and a pharmaceutically suitable carrier. The linked conjugate used in therapeutic applications would be dependent on the condition being treated. For example, if a subject with an inflammatory disorder is being treated, the linked conjugate used would have at least one component, either A or B of Formula I, that has a beneficial effect on the inflammatory disorder being treated.

The pharmaceutical compositions of the present invention include compositions wherein the linked conjugates described herein are present in an effective amount, i.e., in an amount effective to achieve therapeutic (i.e., a therapeutically effective amount) and/or prophylactic benefit (i.e., a prophylactically effective amount). The actual amount effective for a particular application will depend on the patient (e.g. age, weight) the condition being treated; and the route of administration. Determination of an effective amount is well within the capabilities of those skilled in the art, especially in light of the disclosure herein.

The effective amount for use in humans can be determined from animal models. For example, a dose for humans can be formulated to achieve circulating and/or gastrointestinal concentrations that have been found to be effective in animals.

The dosages of the linked conjugates in animals will depend on the disease being, treated, the route of administration, and the physical characteristics of the animal being treated. In some embodiments, the dosage levels of the linked conjugates for therapeutic and/or prophylactic uses can be from about 1 μg/day to about 10 μm/day.

Preferably, the linked conjugates used for therapeutic and/or prophylactic benefits can be administered alone or in the form of a pharmaceutical composition. The pharmaceutical compositions comprise the linked conjugates, one or more pharmaceutically acceptable carriers, diluents or excipients, and optionally additional therapeutic agents. For example, the linked conjugates of the present invention may be co-administered with other active pharmaceutical agents depending on the condition being treated. This co-administration can include simultaneous administration of the two agents in the same dosage form, simultaneous administration in separate dosage forms, and separate administration. In the separate administration protocol, the linked conjugates and the other pharmaceutical agent may be administered a few minutes apart, or a few hours apart, or a few days apart. The linked conjugates can be administered by injection, topically, orally, transdermally, or rectally. Preferably, the linked conjugates or the pharmaceutical composition comprising the linked conjugates is administered orally. The oral form in which the linked conjugates is administered can include powder, tablet, capsule, solution, or emulsion. The effective amount can be administered in a single dose or in a series of doses separated by appropriate time intervals, such as hours.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Suitable techniques for preparing pharmaceutical compositions of the linked conjugates described herein are well known in the art.

### Examples

Having now generally described the invention, the same will be more readily understood through reference to the following examples. The examples are offered for illustrative purposes only, and are not intended to limit the scope of the present invention in any way. Efforts have been made to ensure accuracy with respect to numbers used (e.g., amounts, temperatures, etch), but some experimental error and deviation should, of course, be allowed for.

### Example 1

### The Synthesis of Bromo-Peg-Azide

The compound (2) was synthesized according to the scheme below:

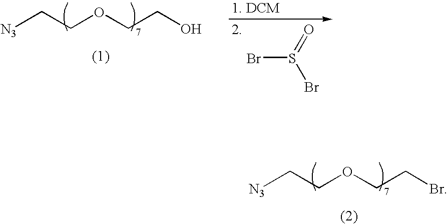

Azido PEG (1) (3 gm, 7.58 mmol, 1 eq) was dissolved in a minimal amount of CH₂Cl₂ (DCM). The mixture was flushed with argon and cooled to 0° C. by ice water bath. Thionyl bromide (1.9 gm, 9.14 mmol, 1.2 eq) was then added drop wise to the mixture. The reaction mixture was stirred at 20° C. for 15 min., warmed to room temperature and stirred overnight. The reaction mixture was poured into a small amount of water and sodium bicarbonate was added to neutralize the mixture to pH of about 7. The mixture was then extracted twice with DCM. The organic layers were combined, washed with brine, dried over MgSO₄, and filtered. The crude mixture was purified by HPLC to obtain product (2).

### Example 2

### Synthesis of Iodo-PEG-Azide

The compound (4) was synthesized according to the scheme below:

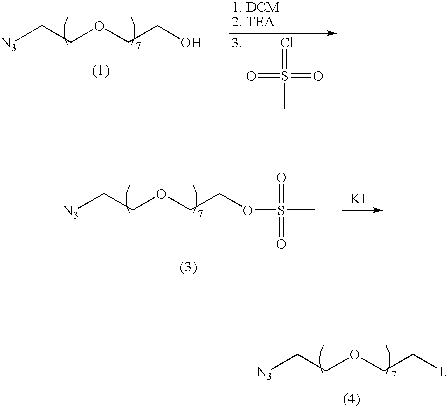

Azido peg (1) (3 gm, 7.58 mmol, 1 eq) was dissolved in a minimal amount of CH₂Cl₂, and triethyl amine (1.53 gm, 15.12 mmol, 2 eq) was added. The reaction was flushed with argon and cooled to 0° C. by ice water bath. Methane sulfonyl chloride (956 mg, 8.35 mmol, 1.1 eq) was added drop wise to the mixture. The reaction stirred at 0° C. for 15 min and then warmed to room temperature. After 4 h, the reaction was stopped by the addition of water, and the aqueous solution was twice extracted with CH₂Cl₂. The organic layers were combined, washed with brine, dried over MgSO₄, filtered, and the organic solvent removed under reduced pressure to yield the intermediate PEG product (3).

The air dried compound (3) was dissolved in a minimal amount of acetone. Potassium iodide (3.15 gm, 18.98 mmol, 2.5 eq) was added to the solution, and the reaction was refluxed overnight at 40° C. under argon. The precipitated salt was removed by filtration, and the filtrate was concentrated under reduced pressure. The concentrated filtrate was diluted with methanol, and purified by HPLC to give the product (4) as a yellowish clear oil.

### Example 3

### Synthesis of Allyl-PEG-Azide

The compound (6) was synthesized according to the scheme below:

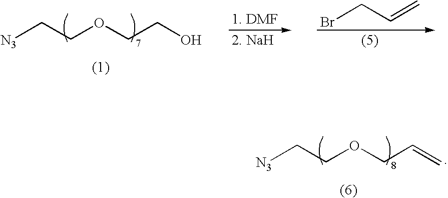

To a stirred suspension of sodium hydride (334 mg, 13.92 mmol, 1.1 eq) in dry DMF at 0° C. under argon was added drop wise a solution of azido peg (1) (5 gm, 12.64 mmol, 1 eq) in dry DMF. The reaction was allowed to warm to room temperature and stirred for 2 h. The reaction solution was cooled to 0° C. by an ice water bath, and a solution of allyl bromide (5) (1.53 gm, 12.65 mmol, 1 eq) in dry DMF was added drop wise. The reaction solution was allowed to warm to room temperature, stirred overnight, and the salt precipitate was removed by filtration. The filtrate was purified by HPLC to yield the product (6) as a light yellow oil.

### Example 4

### 6-(6-tert-Butoxycarbonylamino-hexanoylamino)-hexanoic acid 2,5-dioxo-pyrrolidin-1-yl ester

The compound (15) was synthesized according to the scheme below:

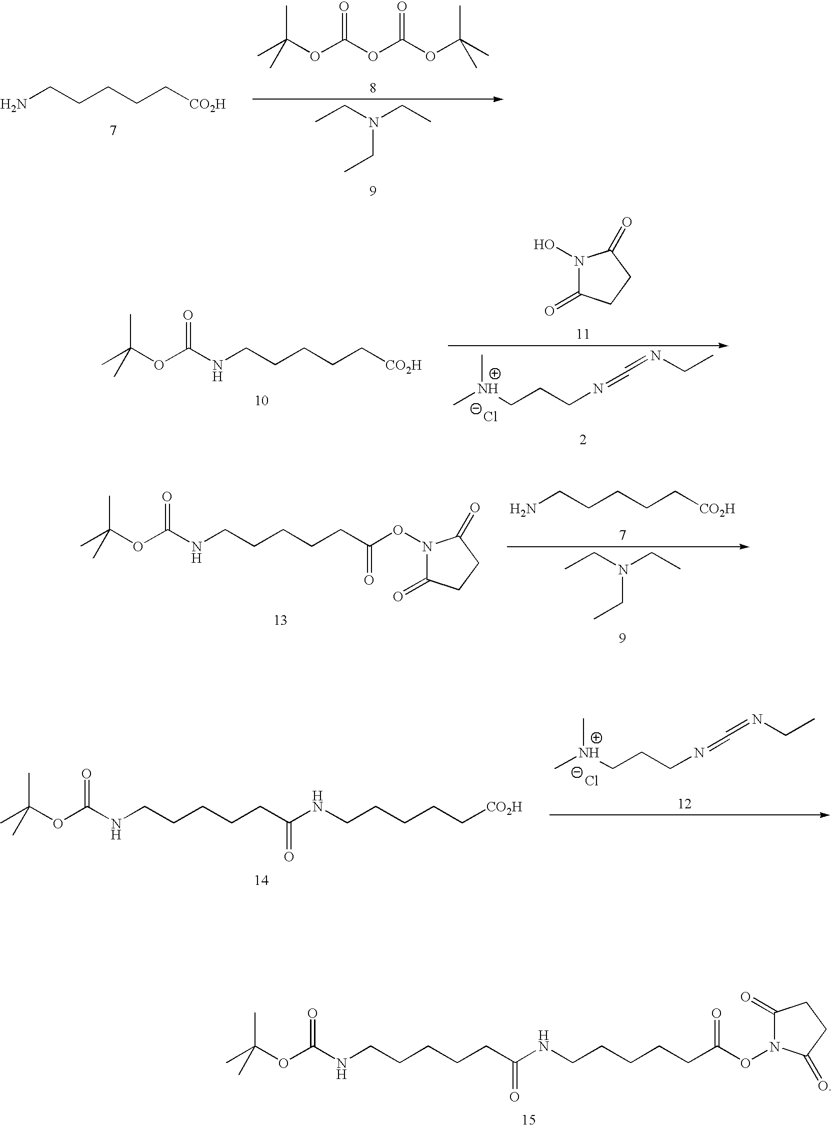

Aminocaproic acid (7) (16.39 g, 0.1249 mol, 1 equiv.) was suspended in 150 mL dimethylacetamide. Triethylamine (9) was added (22 mL, 0.1578 mol, 1.26 equiv.), followed by di-tert-butyl dicarbonate (8) (33.42 g, 0.1531 mol, 1.23 equiv.). The mixture was vigorously stirred at room temperature until the solid 7 dissolved and the solution was clear and homogeneous (about 18 h). Solid N-hydroxysuccinimide (11) (16.36 g, 0.1422 mol, 1.138 equiv.) was added, followed by more triethylamine (9) (23 mL, 0.165 mol, 1.32 equiv.), and solid (3-dimethylaminopropyl)-ethylcarbodiimide (12) (36.73 g, 0.1916 mol, 1.53 equiv.), and 20 mL of dimethylacetamide. The heterogeneous mixture was stirred overnight (18 h). A milky, nearly clear solution resulted. Solid 6-aminocaproic acid (7) (16.35 g, 0.1246 mol, 1 equiv.) and 50 mL of dimethylacetamide were added, after which the mixture was heated to 60° C. for 30 minutes. More (3-dimethylaminopropyl)-ethylcarbodiimide (12) (36.89 g, 0.1924 mol, 1.54 equiv.), was added, followed by 30 mL of dimethylacetamide. The reaction mixture was stirred at 50° C. overnight. The resulting white precipitate (a triethylamine salt) was filtered off, washed with more dimethylacetamide, and discarded. The combined dimethylacetamide layers were diluted with water (1 L) and citric acid monohydrate (3 g) were added until the pH was about 5. The mixture was extracted with ethyl acetate (3×150 mL). The combined ethyl acetate extracts were washed with water and brine, dried over Na2SO4 and concentrated to a pasty light green solid 15: 36.38 g (82.4 mmol, 66%).

### Example 5

### 6-(6-tert-Butoxycarbonylamino-hexanoylamino)-hexanoic acid N-{2-[2-Azidoethoxy-octakis(2-ethoxy)]ethyl}amide

The compound (17) was synthesized according to the scheme below:

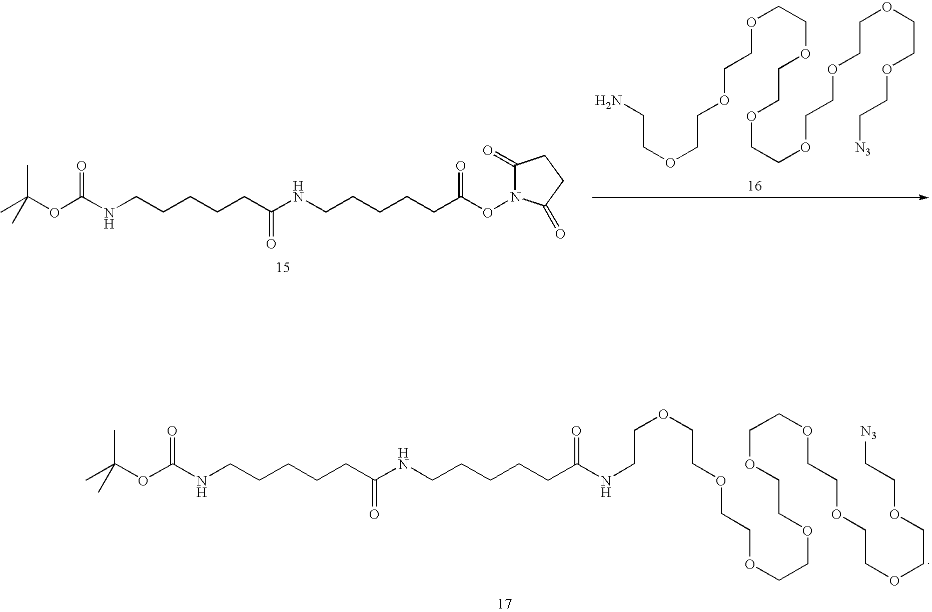

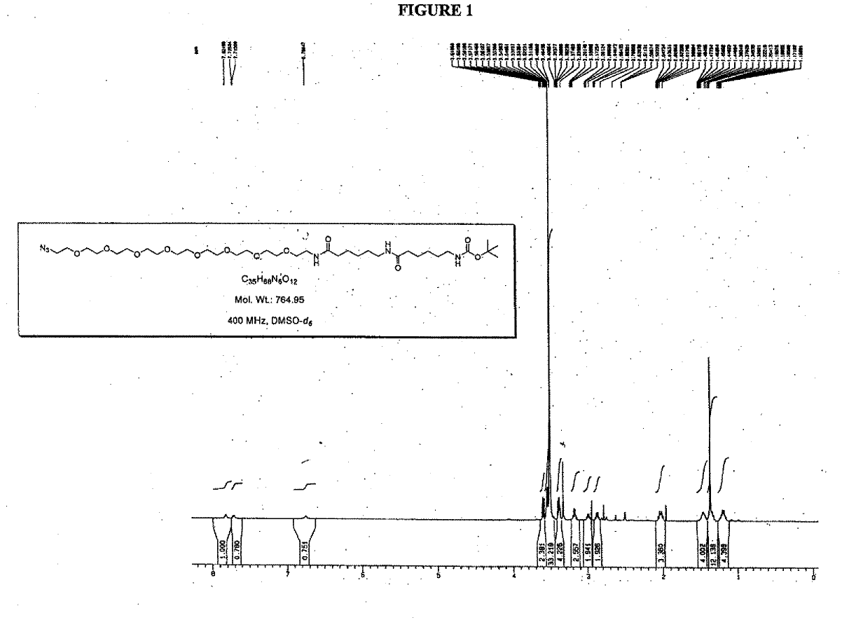

6-(6-tert-Butoxycarbonylamino-hexanoylamino)-hexanoic acid 2,5-dioxo-pyrrolidin-1-yl ester (15) (13.07 g, 29.6 mmol, 1.05 equiv.), 2-[2-Azidoethoxy-octakis(2-ethoxy)]ethylamine (16) (12.4 g, 28.3 mmol, 1 equiv.), and triethylamine (9) (5.0 mL, 35.77 mmol, 1.27 equiv.) were dissolved in 120 mL of chloroform. The mixture was heated to 55° C. for 3.5 h. The reaction was not complete until (3-dimethylaminopropyl)-ethylcarbodiimide (12) (˜1 g) was added. The mixture was cooled to room temperature. It was diluted with 300 mL chloroform and washed with 50 mL of water (the emulsion slowly separated). Drying over Na₂SO₄ and concentrating yielded 17 as a waxy solid: 20.55 g (26.86 mmol, 95%). NMR of the compound is provided in FIG. 1.

### Example 6

### 6-(6-Amino-hexanoylamino)-hexanoic acid N-{2-[2-Azidoethoxy-octakis(2-ethoxy)]ethyl}amide

The compound (19) was synthesized according to the scheme below:

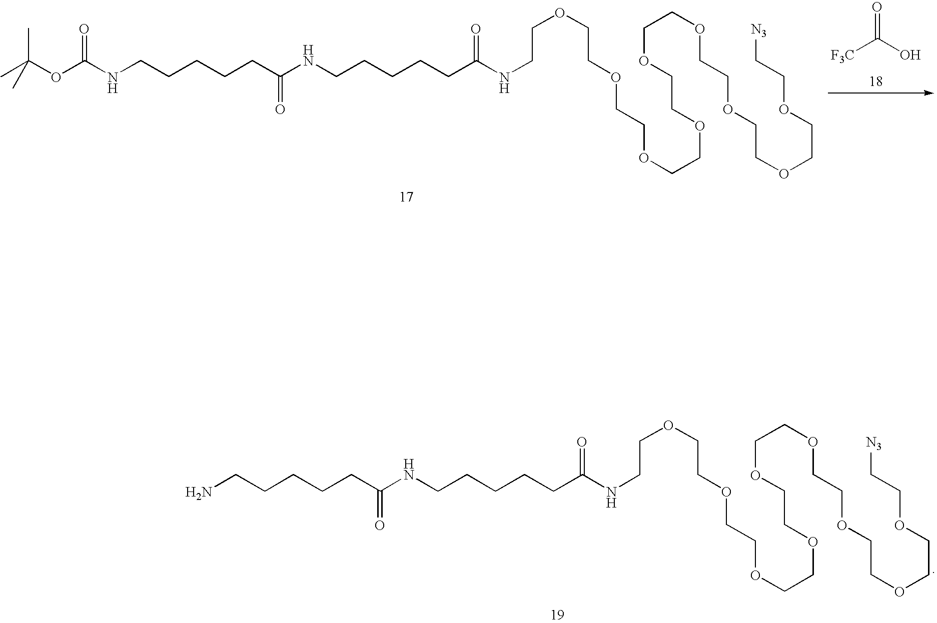

6-(6-tert-Butoxycarbonylamino-hexanoylamino)-hexanoic acid N-{2-[2-Azidoethoxy-octakis(2-ethoxy)]ethyl}amide (17) (20.55 g, 26.86 mmol, 1 equiv.) was dissolved in 100 mL dichloromethane and 35 mL trifluoroacetic acid. After 4 hours, no more 6 could be detected by LCMS. Concentration to a glassy solid, addition of 2 mL of water and solid K₂CO₃ until no more effervescence occurred, were followed by extracting with chloroform. Drying over Na₂SO₄ and concentration yielded an oil. Further concentration on high-vacuum yields a glassy solid product 19. Crude yield: 24.67 g (quantitative). It was used as such in the next step.

### Example 7

### 6-[5-(2-Oxo-hexahydro-thieno[4-d]imidazol-4-yl)-pentanolamino]-hexanoic acid [5-{2-(2-amino-ethoxy)-octakis(2-ethoxy)}-ethylcarbamoyl)-pentyl-amide

The compound (22) was synthesized according to the scheme below:

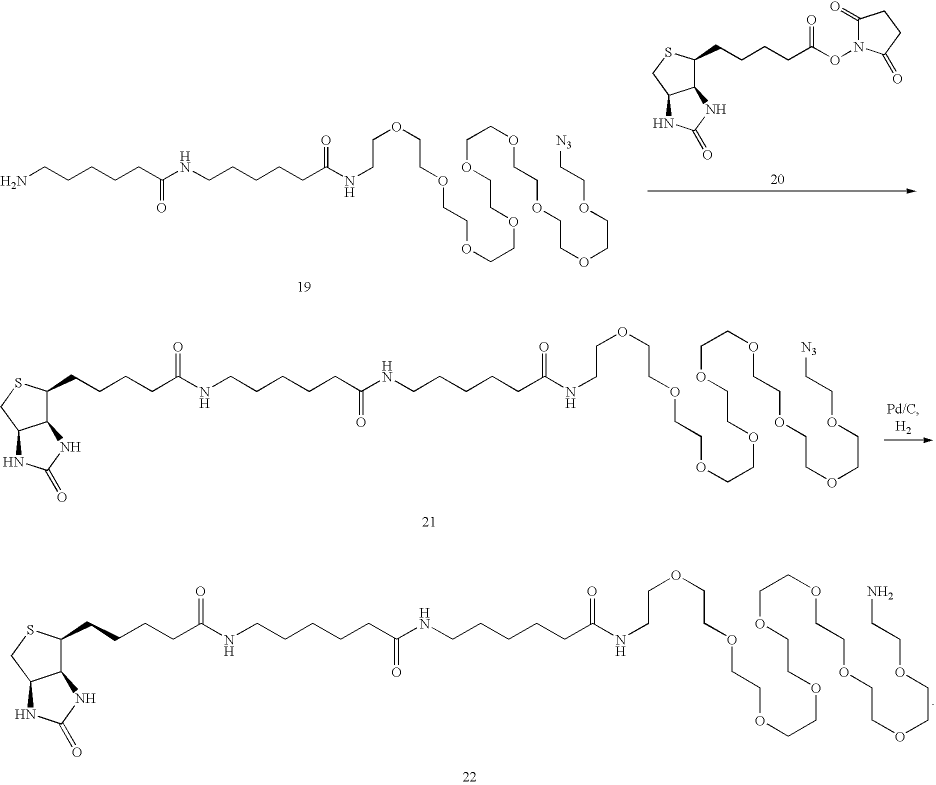

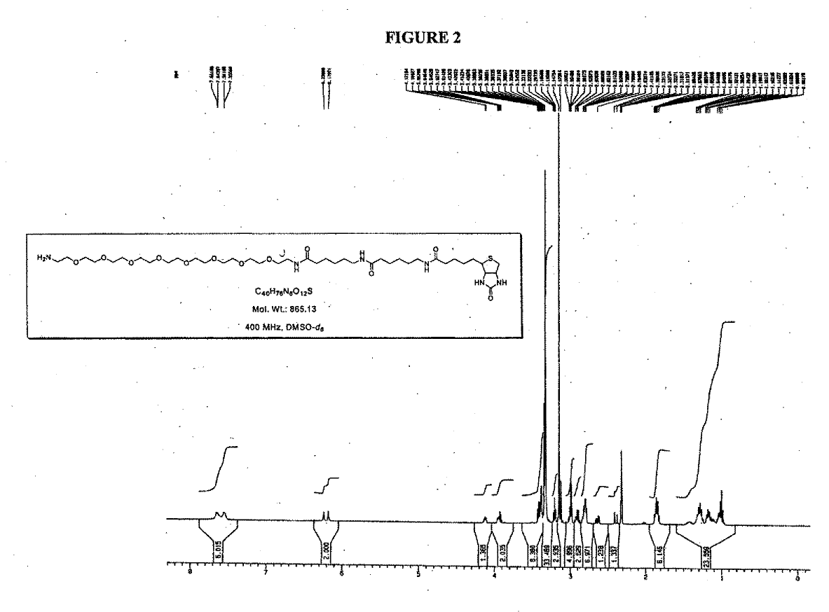

The crude product 19 from the previous procedure (26.86 mmol, 1 equiv.) was dissolved in 120 mL chloroform and 18 mL triethylamine (129 mmol, 4.77 equiv.). 5-(2-Oxo-hexahydro-thieno[3,4-d]imidazol-4-yl)-pentanoic acid 2,5-dioxo-pyrrolidin-1-yl ester (20) (9.22 g, 27 mmol, 1 equiv.; for preparation see below) was added and the mixture was heated to 55° C. for 50 minutes. The mixture was cooled, diluted with more chloroform, and extracted with water. The chloroform solution was dried over Na₂SO₄ and concentrated to a waxy solid that contained also triethylammonium trifluoroacetate. Crude yield of compound 21 was 31 g. The crude azide 21 (26.86 mmol, 1 equiv.) was dissolved with heating in 220 mL methanol. The solution was degassed with argon and palladium black (10% on carbon; 1.78 g, 1.67 mmol, 6.2%) was added. The mixture was vigorously stirred under hydrogen gas (1 atm.) for 2 hours. LCMS indicates no more azide 21 was present. Filtration and concentration, followed by silica gel chromatography (dichloromethane to 15% methanol in dichloromethane to 40% methanol in dichloromethane) yields pure 22 as a glass: 10.63 g (12.3 mmol, 46%, 3 steps). The NMR of the compound is given in FIG. 2.

### Example 8

### 5-(2-Oxo-hexahydro-thieno[3,4-d]imidazol-4-yl)-pentanoic acid 2,5-dioxo-pyrrolidin-1-yl ester

The compound (20) was synthesized according to the scheme below:

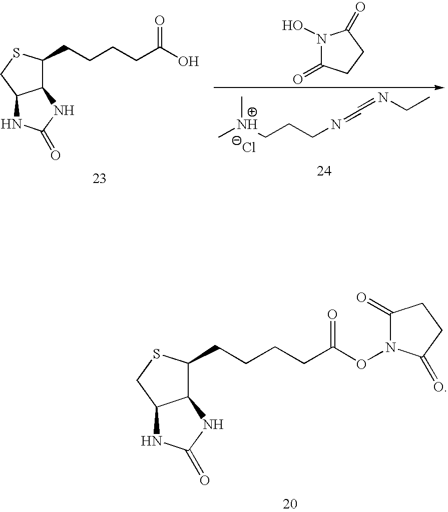

Biotin (23) (17.37 g, 71.1 mmol, 1 equiv.) was dissolved in 100 mL dimethylacetamide. N-Hydroxysuccinimide (9.06 g, 78.7 mmol, 1.10 equiv.) and triethylamine (20 mL, 143.5 mmol, 2.02 equiv.) were added, followed by (3-dimethylaminopropyl)-ethylcarbodiimide (24) (18.25 g, 95.2 mmol, 1.34 equiv.). The milky solution was stirred at room temperature for 18 hours. A fine white precipitate formed. The mixture was diluted with 50 mL of water and the fine precipitate was filtered using a fine porosity scintered glass filter, was washed with more water and dried to yield 20 as a white, free-flowing solid: 17.43 g, 51.1 mmol, 72%.

### Example 9

### Preparation of Alkyne-Functionalized LCLC-Biotin

The alkyne-functionalized LC-LC-biotin compound (25) was synthesized according to the scheme below:

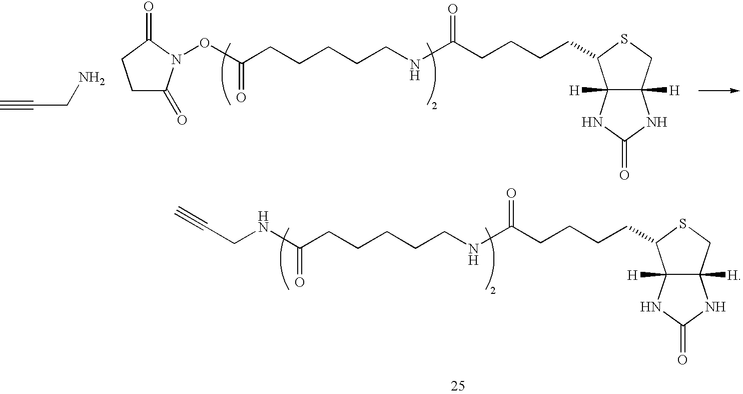

Propargylamine (400 μL, 5.8 mmol) was added to a solution of biotin-LCLC-O-succinimide (1.0 g, 1.7 mmol) in chloroform and stirred at room temperature over night. Purification by HPLC yielded pure biotin-LCLC-propargylamide.

### Example 10

### Linking of Purvalanol B

Purvalanol B (26) was linked to give the product 27 according to the scheme below:

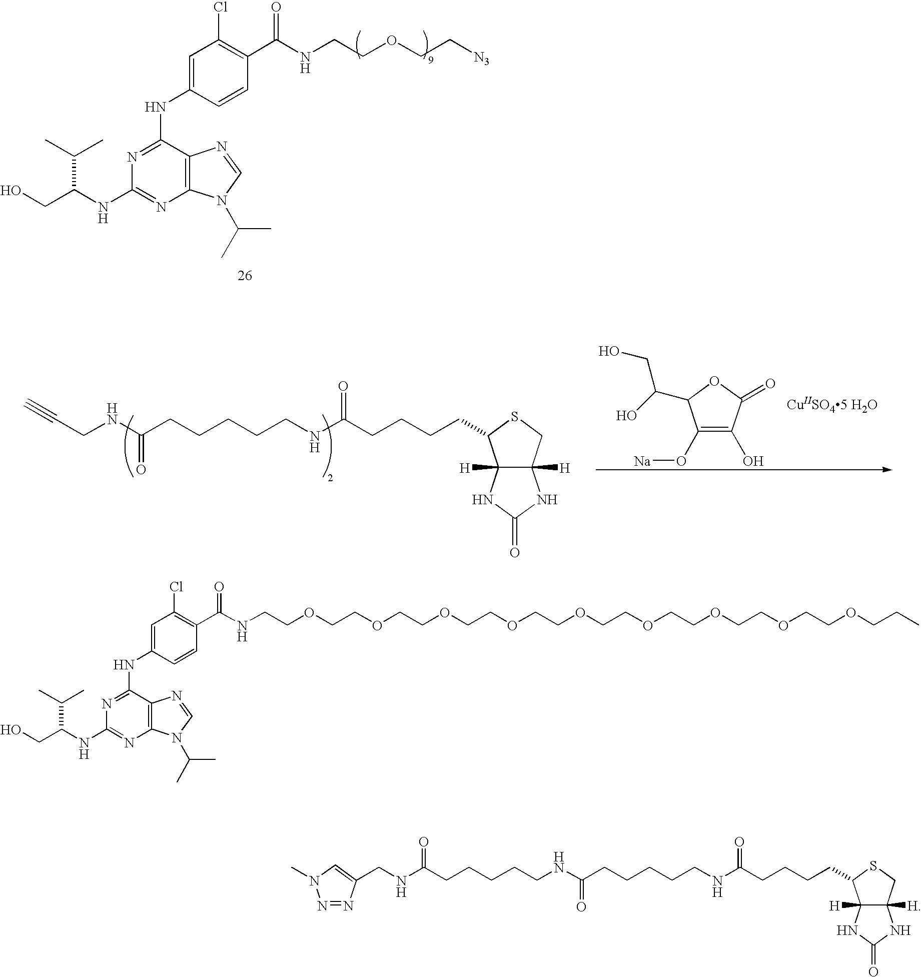

To a mixture azido-PEG-Purvalanol B (15 mg, 16.7 μmol) and biotin-LC-LC-propargylamide (8.5 mg, 16.7 μmol) in 1 mL tert-butanol/water (1:1) was added 0.1 eq. of aqueous sodium ascorbate solution, followed by 0.01 eq. of aqueous copper(II)sulfate solution. The mixture was stirred at room temperature over night. Purification by HPLC yielded the pure biotin-conjugate of Purvalanol B (27).

Similarly, the amine-linker (28) below:

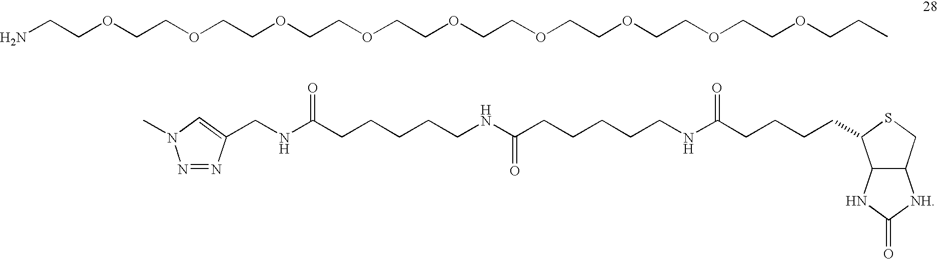

was prepared from the azido-PEG amine and biotin-LC-LC propargylamide using the procedure detailed above.

### Example 11

### Linking of SB 202190

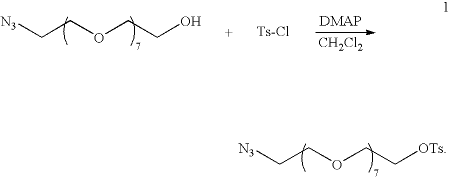

1.0 g Azido-PEG-OH was mixed with 0.8 g tosyl chloride and 0.5 g DMAP in 15 mL of dichloromethane (DCM) and stirred at room temperature over night. The reaction was quenched with 50 ml of 1N HCl and extracted with 3×20 mL DCM. The combined organic phases were dried over MgSO4, filtered, and evaporated. Chromatography over silica yielded (93%) TsO-PEG-azide as a colorless oil.

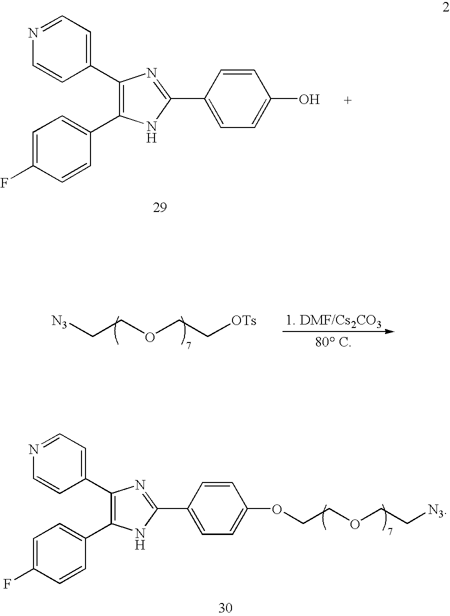

0.5 g (1 eq.) of SB 202190 (29) was dissolved in 5 mL dry DMF. To this solution was added 0.912 g (1.1 eq.) N₃-7PEG-OTs prepared above and 0.540 g (1.1 eq.) of cesium carbonate. The reaction mixture was stirred at 80° C. for 24 h, than evaporated under vacuum to give 1.06 g of the crude azide. The azide product was purified by preparative HPLC to give the purified product (30) with a 80% yield.

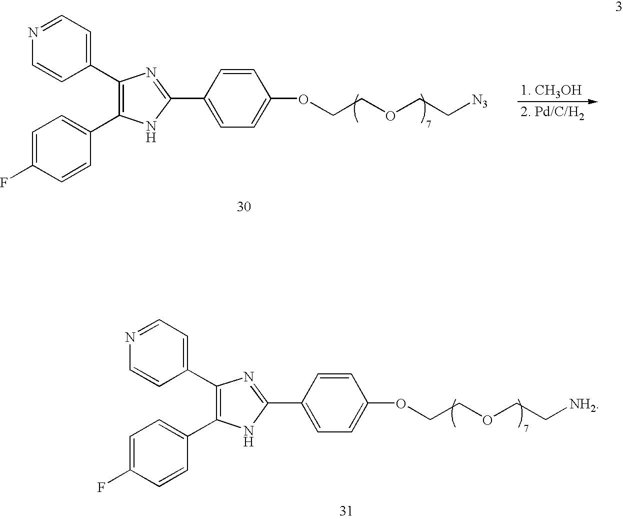

0.8 g of the azide product (30) was dissolved in 5 ml methanol and 0.1 g 10% Pd/C was added. The reaction mixture was stirred under hydrogen for 8 h, at the end of which starting material was not detected by TLC. The methanol solution was filtered trough Celite and evaporated to dryness to give the crude primary amine (31) with an 88% yield. The primary amine (31) which was used without purification in the next step.

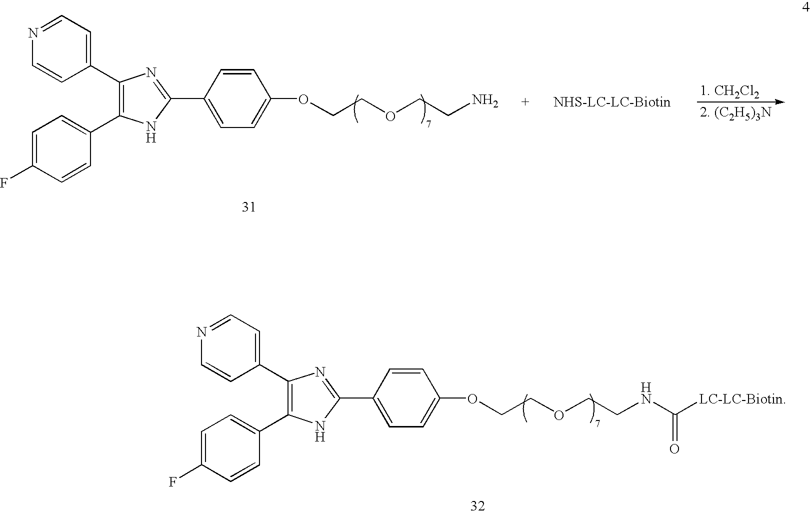

0.6 g of the primary amine (33) prepared in the previous step and 0.548 g (1.1 eq.) NHS-LC-LC-biotin was dissolved in 5 mL of dichloromethane and 0.1 mL of triethylamine was added. The suspension was stirred at room temperature until a clear solution was obtained (approximately 24 h), than the solvent evaporated in vacuum to give 0.997 g of the crude product (32). The crude was purified by preparative HPLC to give the final product (32) in a 71% yield.

Using the procedures described above, compounds 33 to 74, shown below, were made.

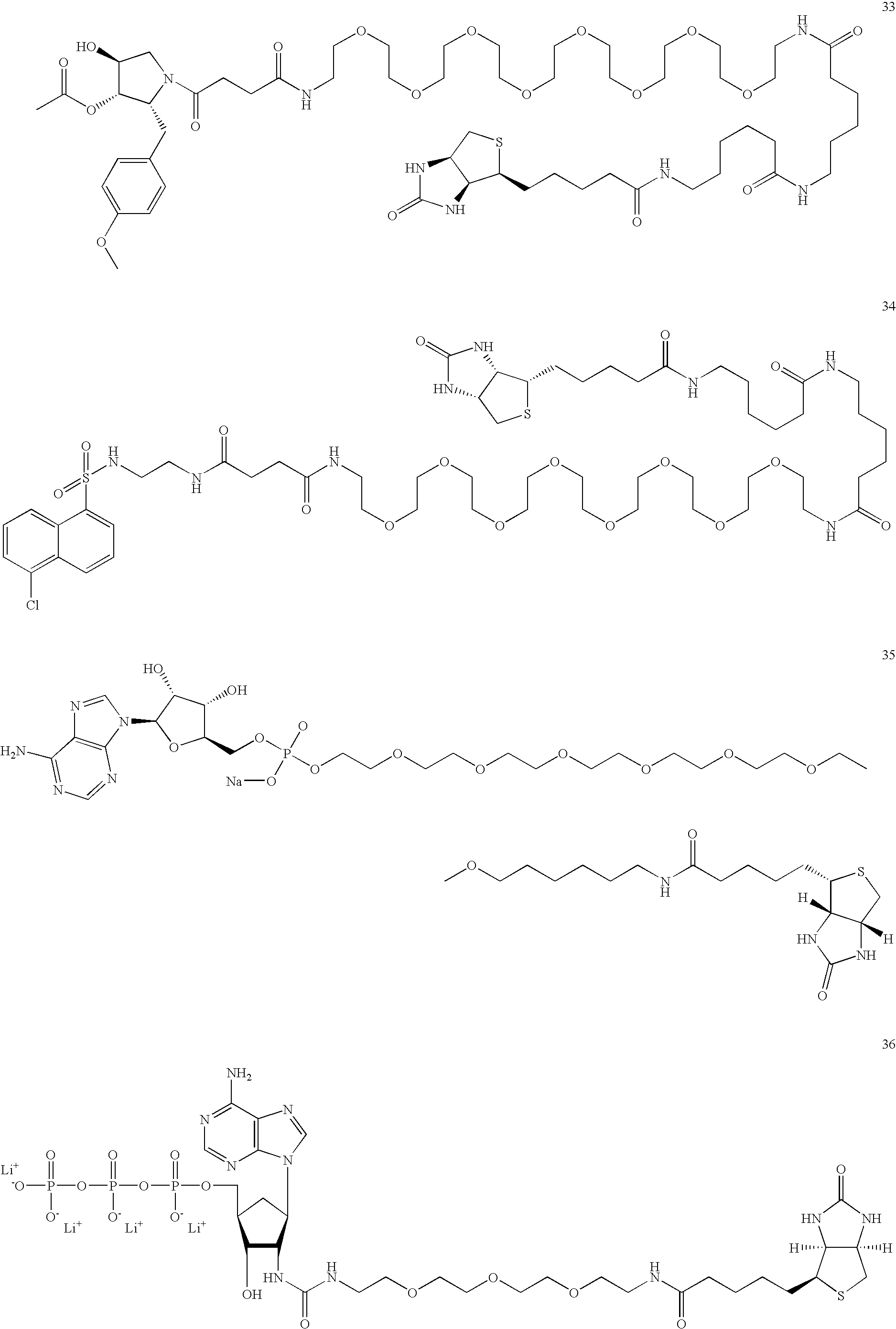

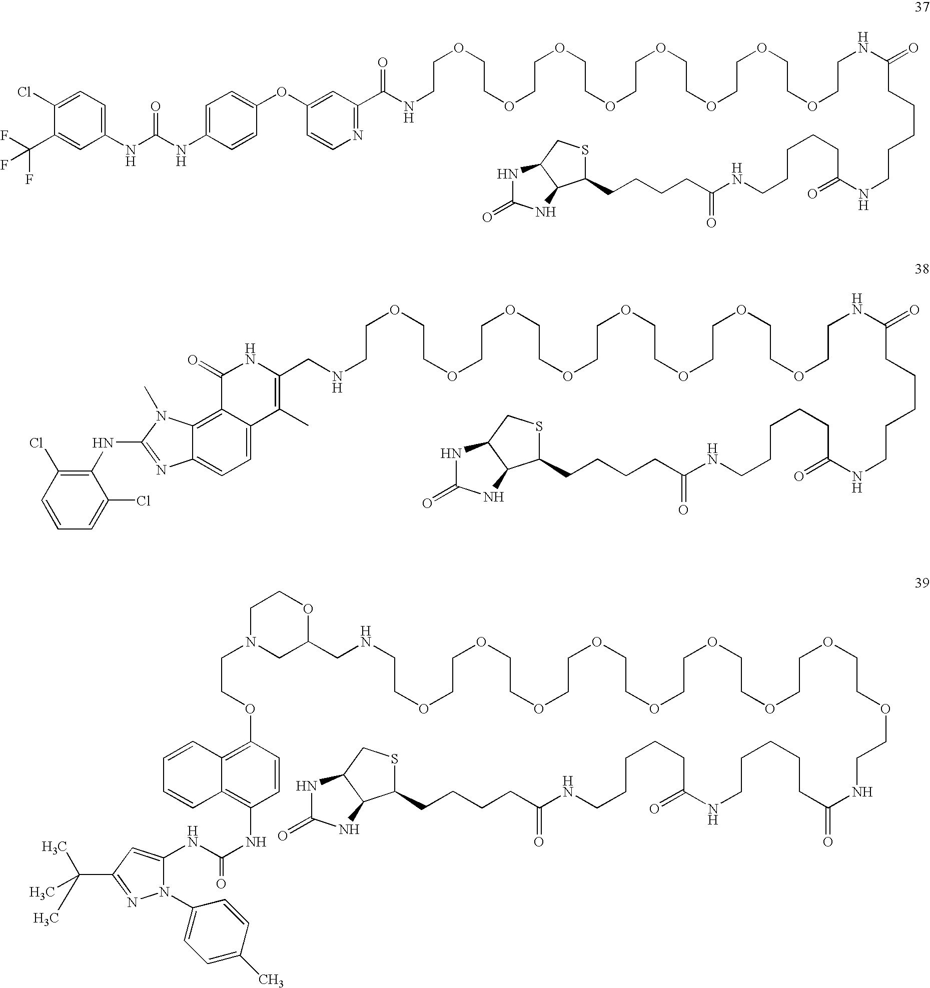

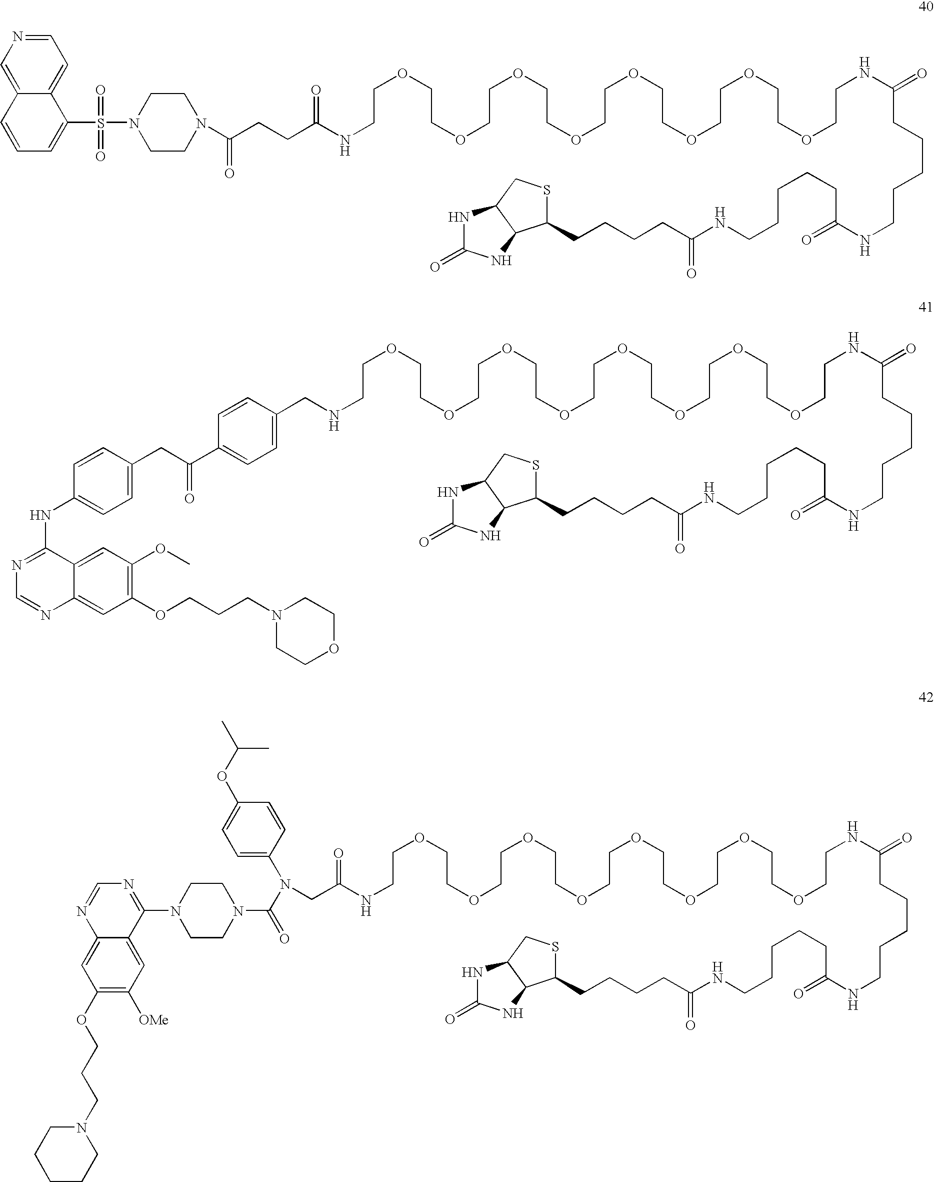

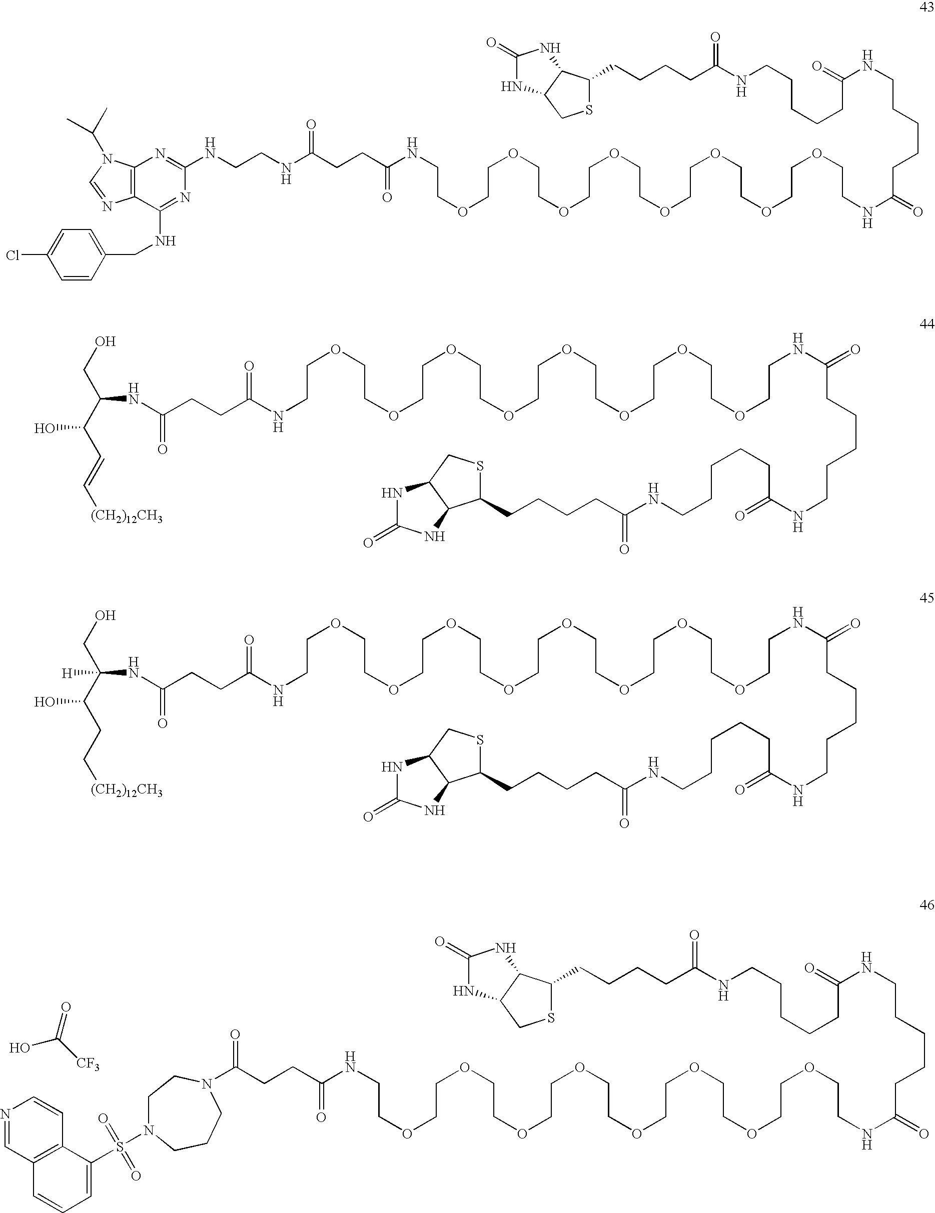

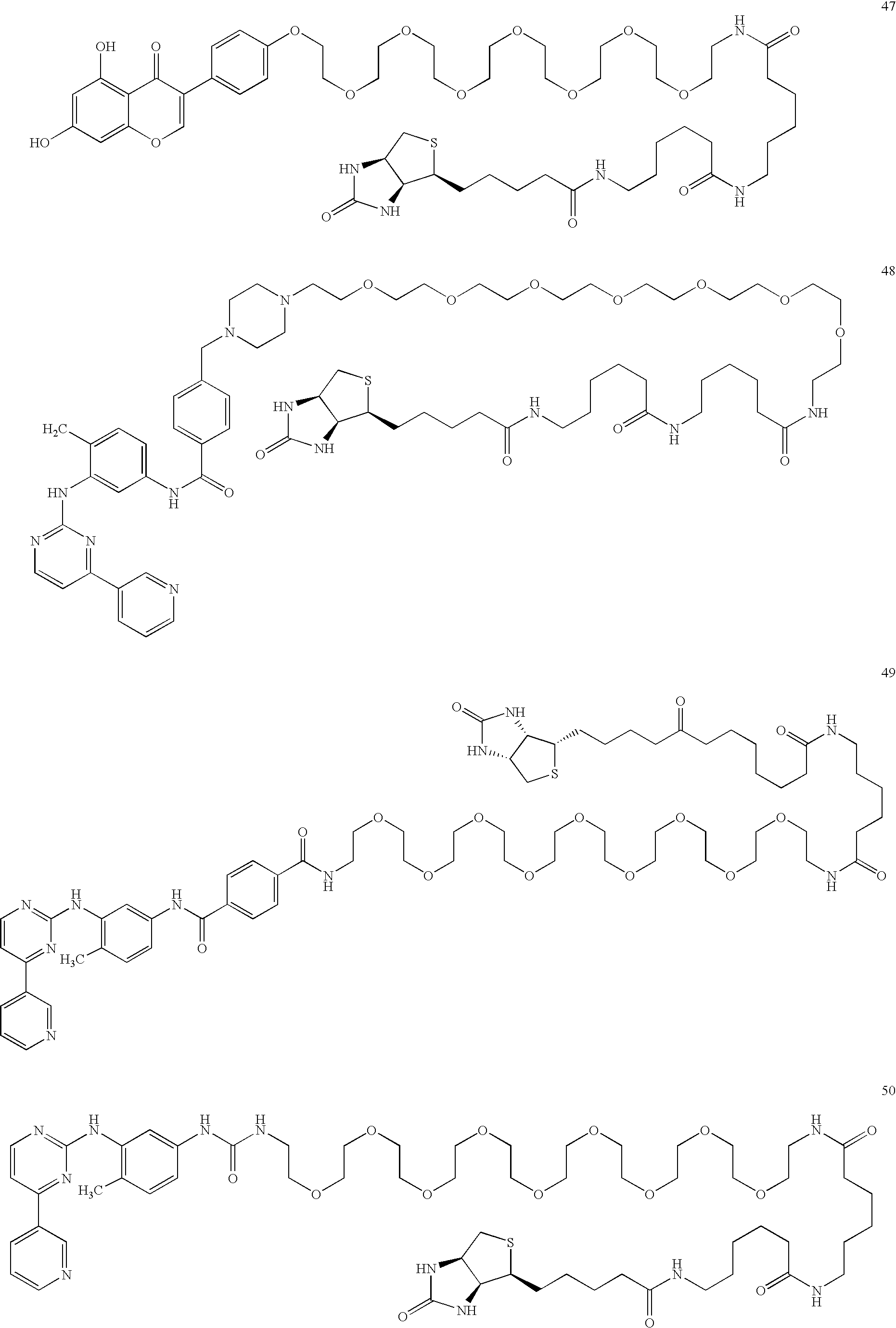

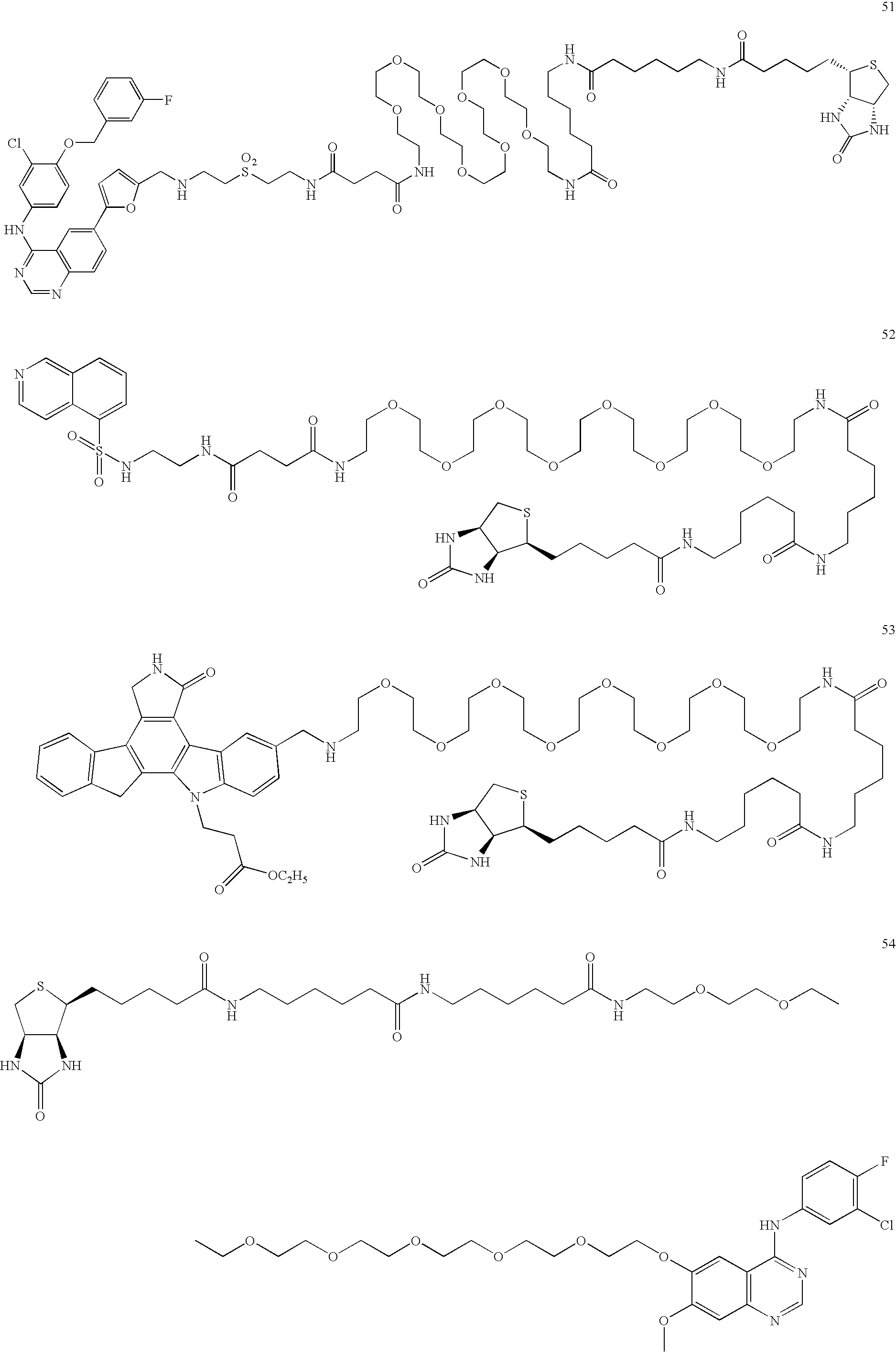

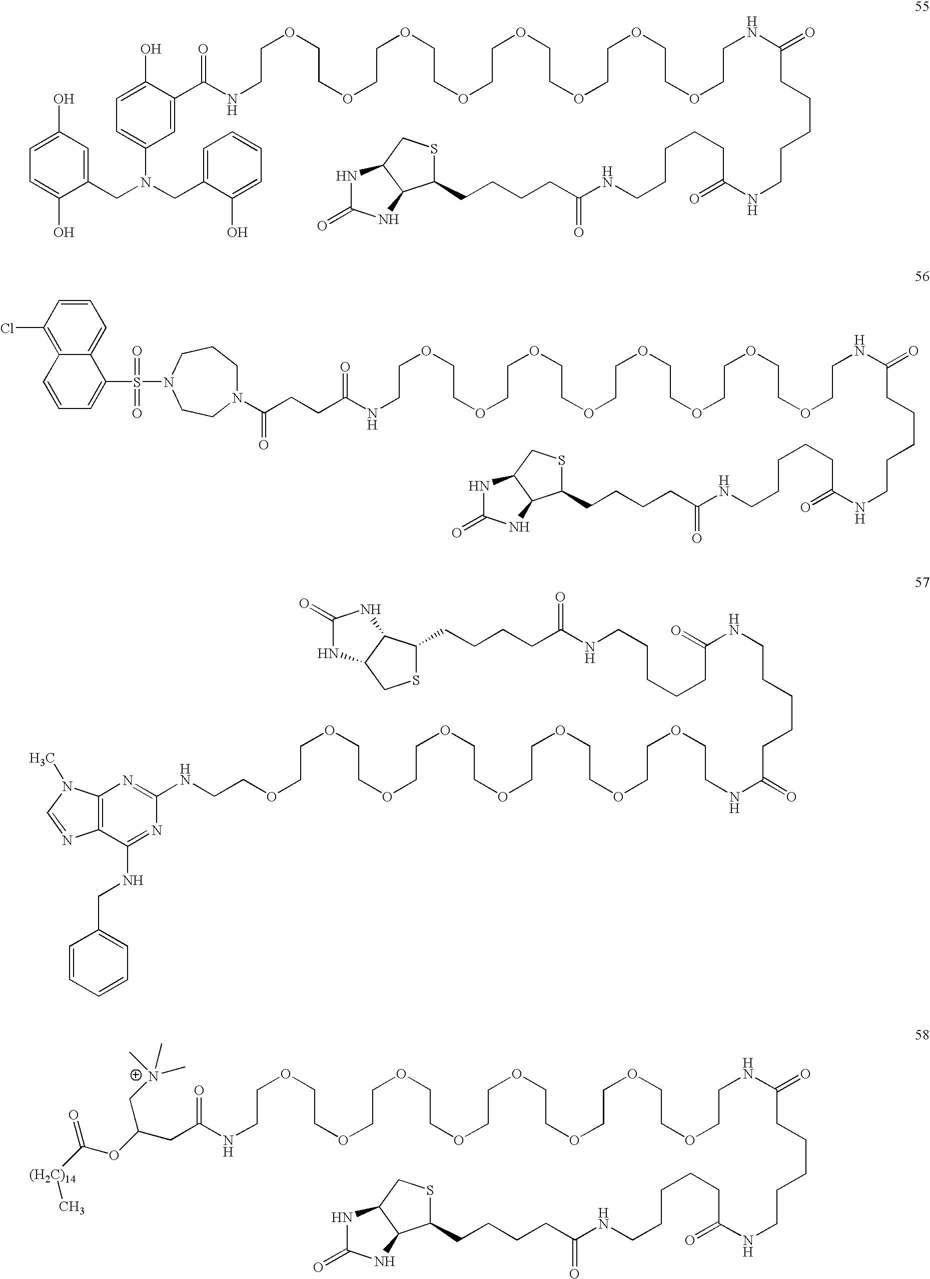

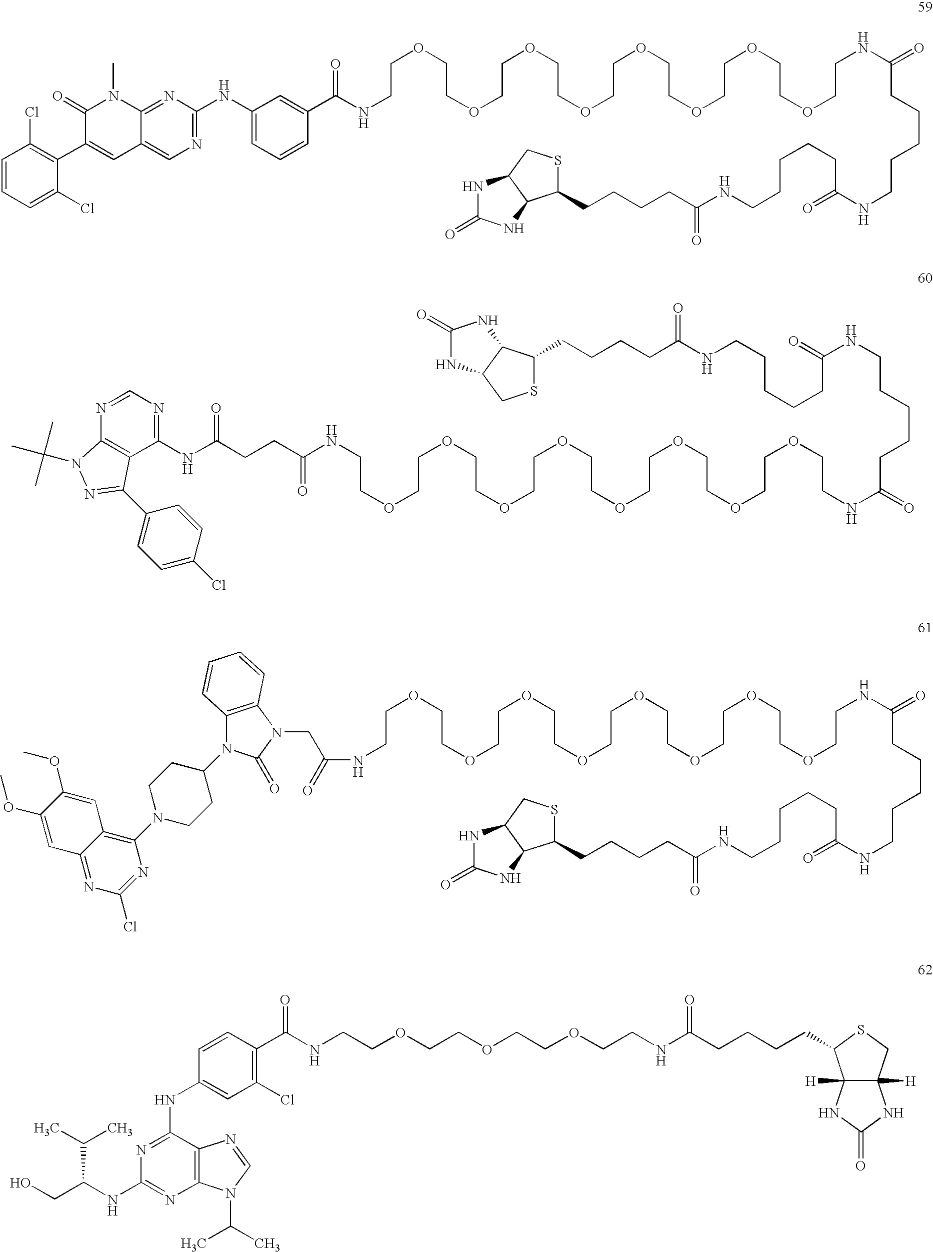

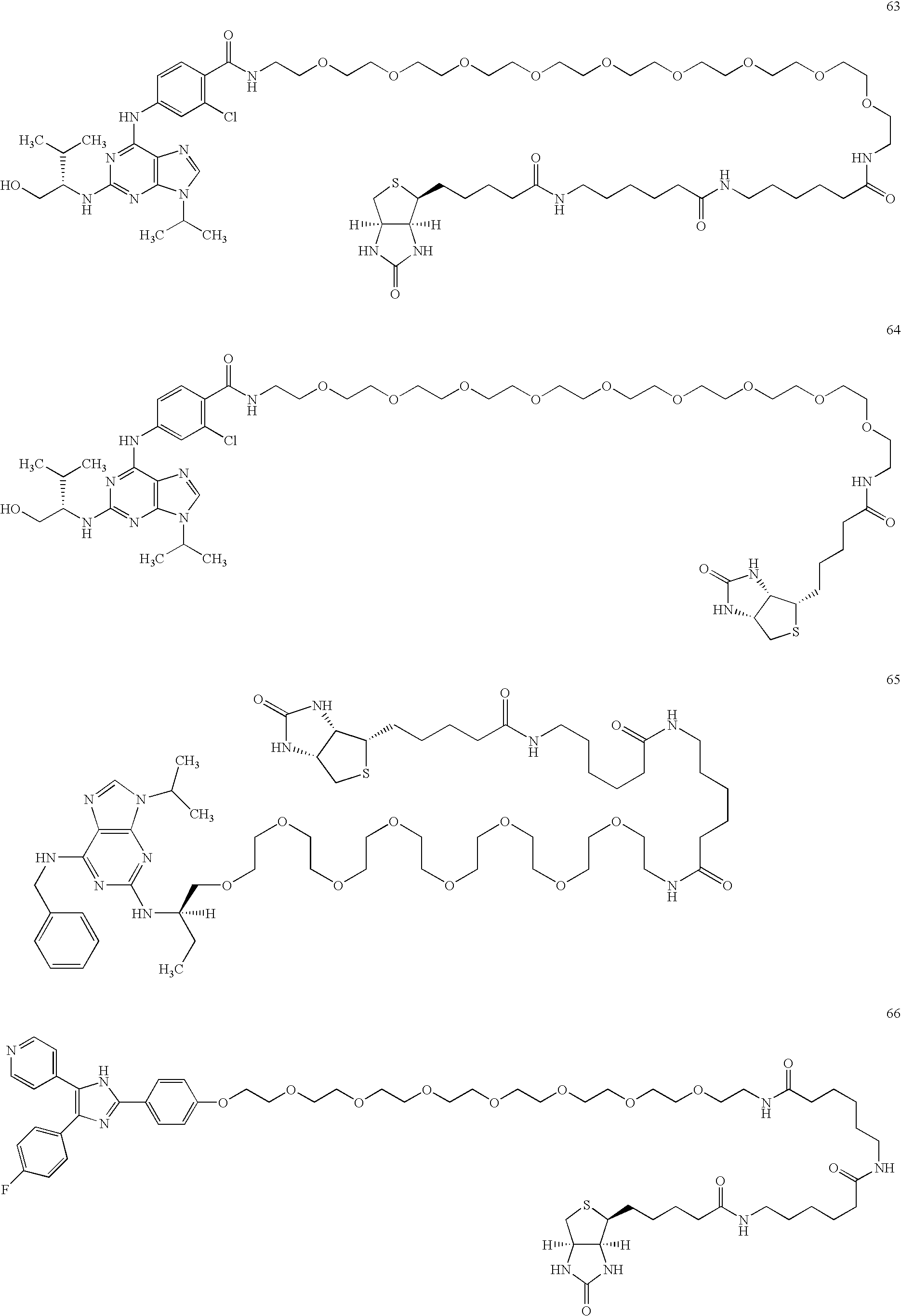

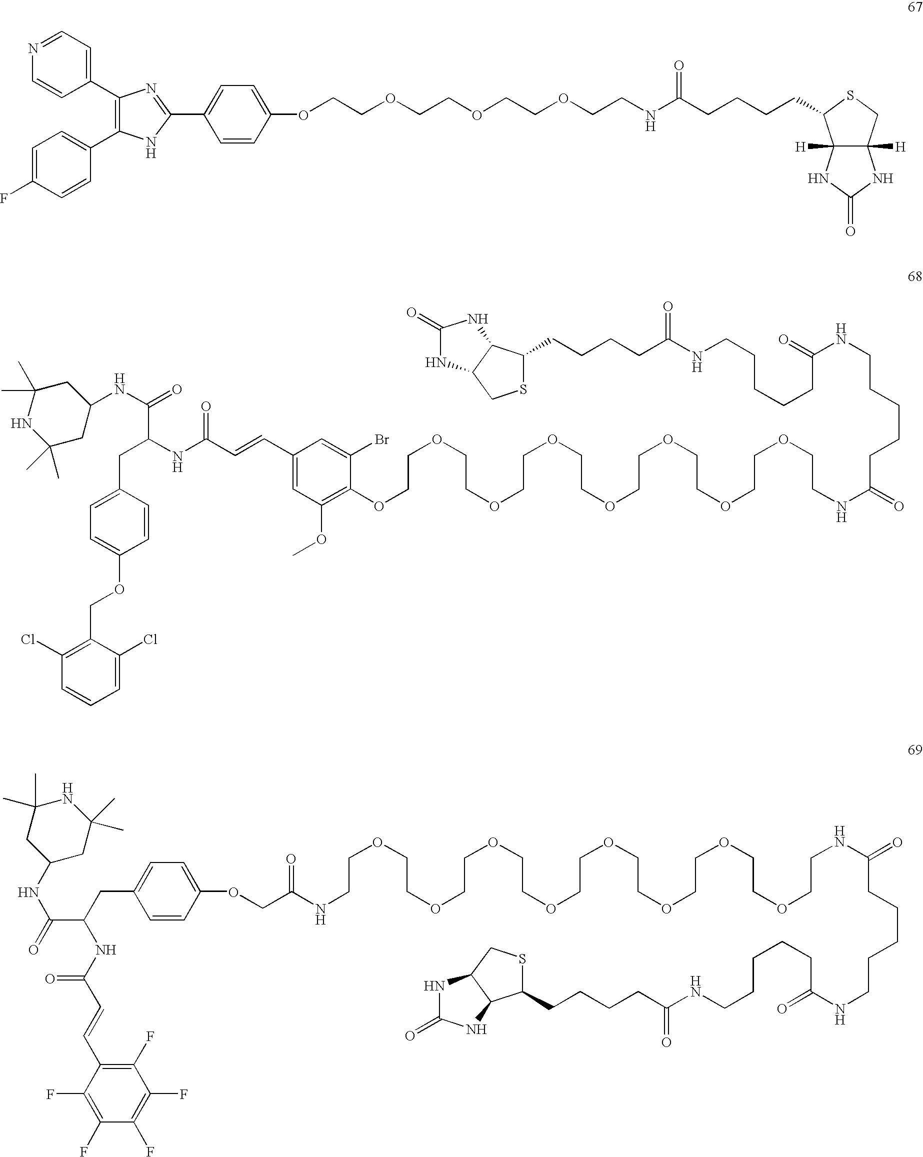

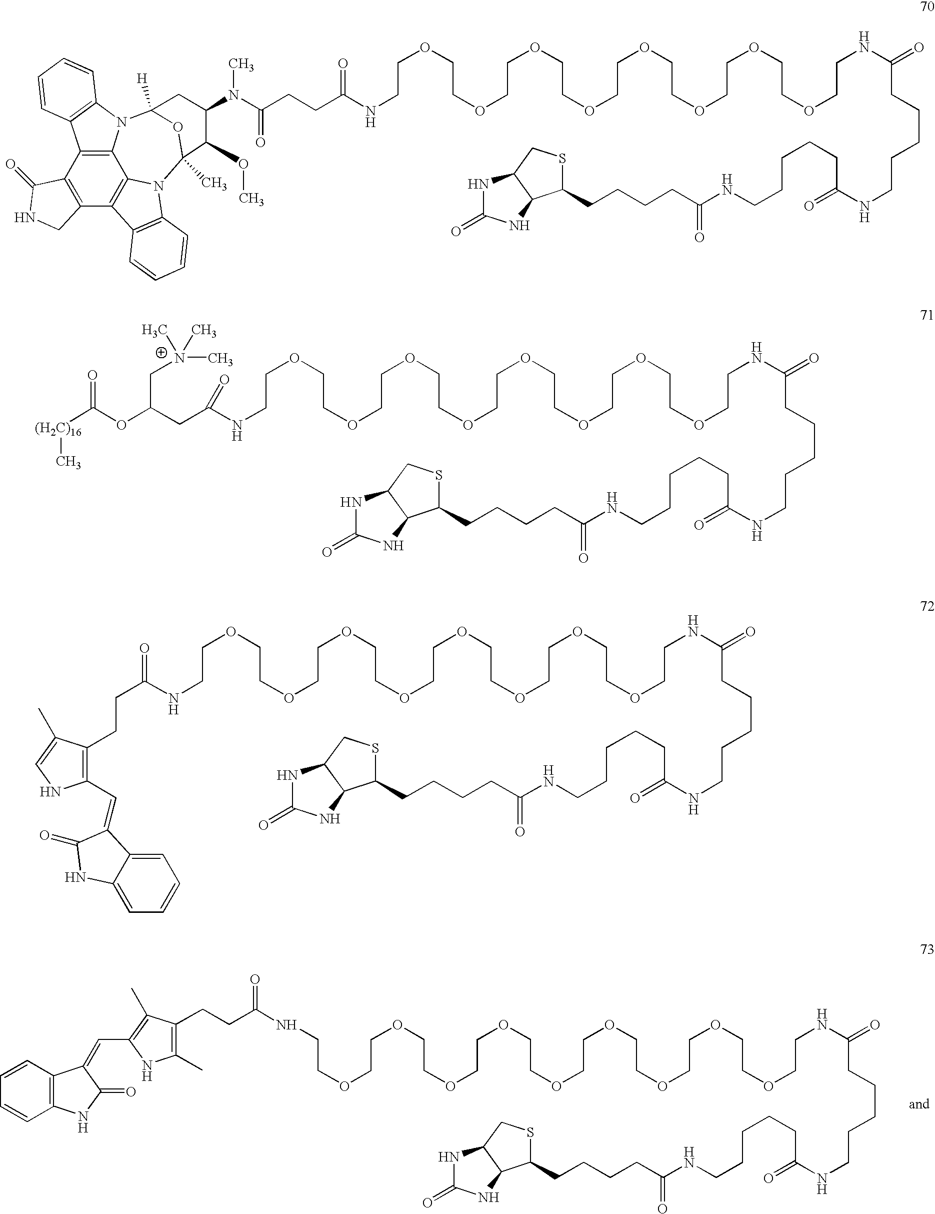

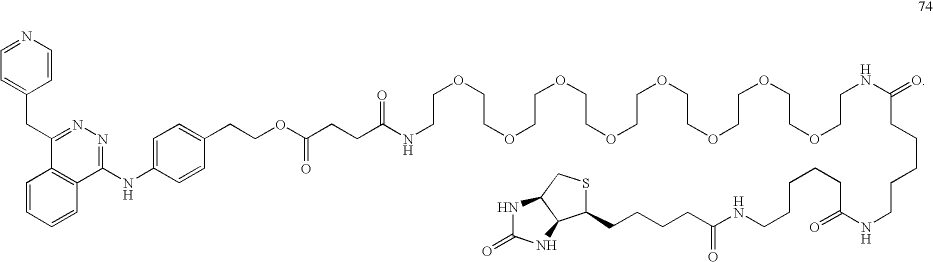

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A compound having a formula:
A-(X₁—(CR₁R₂)ₚ—C(═X₂))ₙ—X₃-T₁-(CR₃R₄X₄CR₅R₆)ₘ-T₂-L-B (FORMULA A1)
wherein:
B is selected from: 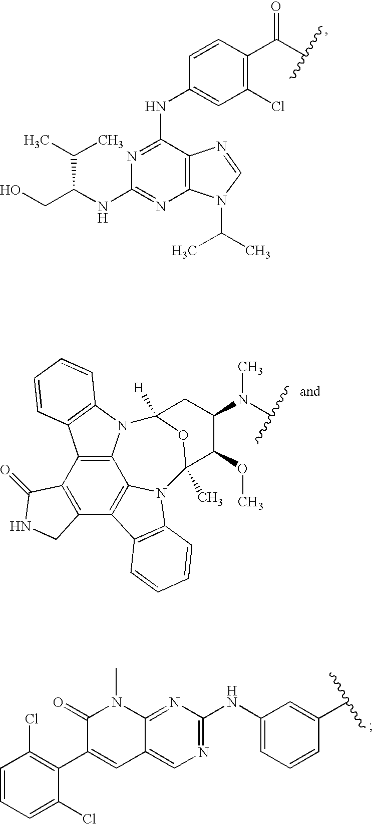
A is biotin, a biotinyl group or a derivative of a biotinyl group;
X₁, X₂, X₃, and X₄ are independently selected from the group consisting of O, S, and NH;
R₁, R₂, R₃, R₄, R₅, R₆, R₇, and R₈, are each independently selected from a group consisting of hydrogen, halogen, or lower alkyl;
T₁ and T₂ are each independently selected from a group consisting of (CH₂)ᵣ, a bond or triazole;
L is a bond or a moiety comprising a —NH— group;
p is 1, 2, 3, 4, 5, or 6;
n is 0, 1, 2, 3, 4, 5, or 6;
r is 1, 2, 3, 4, 5, or 6; and
m is 4 to 20.

2. The compound of claim 1, wherein L is a moiety comprising an —NH—C(O)— group.

3. The compound of claim 1, wherein L is selected from the group consisting of —NH—, —C(O)NH—, —NHC(O)NH—, and —C(O)CH₂CH₂C(O)NH—.

4. The compound of claim 1, wherein p is 5.

5. The compound of claim 1, wherein p is 5; n is 1, 2, or 3; and m is 6, 7, 8, 9, or 10.

6. The compound of claim 1, wherein A is:

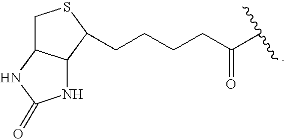

7. The compound of claim 1, wherein X₂ is O.

8. The compound of claim 1, wherein X₄ is O.

9. The compound of claim 1, wherein T₂ is CH₂.

10. The compound of claim 1, wherein T₁ is triazole.

11. The compound of claim 1 selected from:

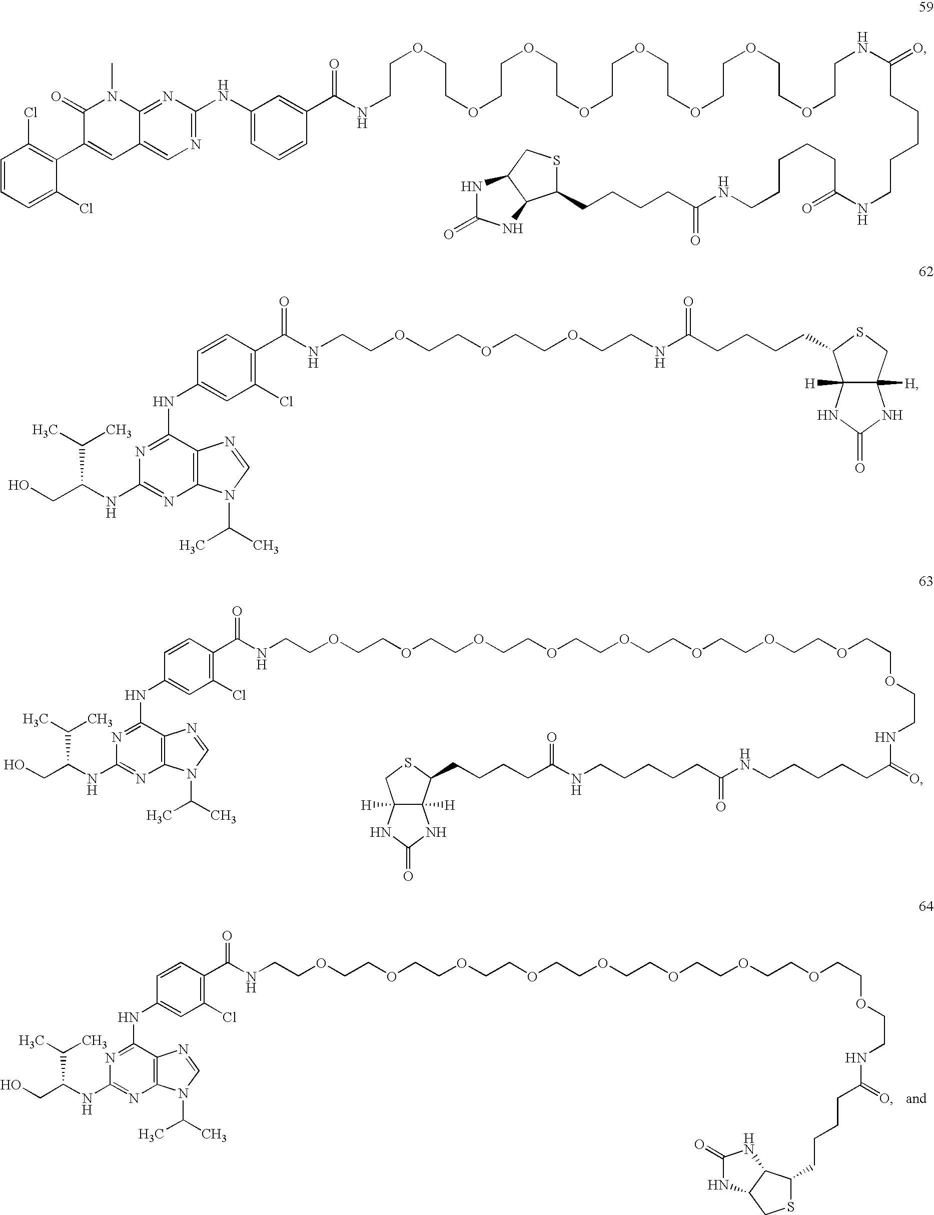

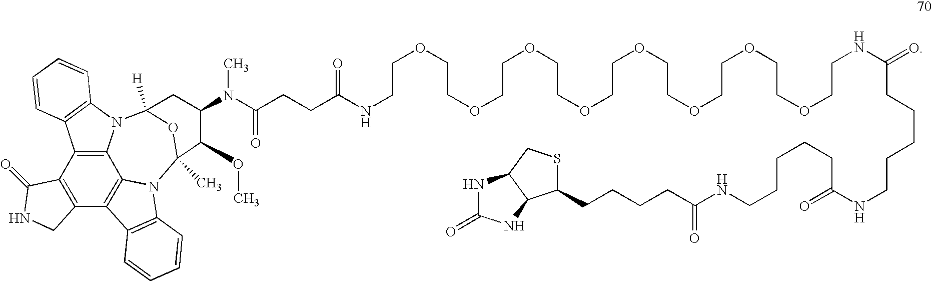

12. A compound having formula: [chemical structure]
